(19) United States
(12) Patent Application Publication
(10) Pub. No.: US 2026/0139308 A1
(43) Pub. Date: May 21, 2026

(54) OPTIMISED SET OF OLIGONUCLEOTIDES FOR BULK RNA BARCODING AND SEQUENCING

(71) Applicant: ALITHEA GENOMICS SA, Cossonay-Ville (CH)

(72) Inventors: Daniel ALPERN, Evian-les-bains (FR); Riccardo DAINESE, Cossonay-Ville (CH)

(21) Appl. No.: 18/866,619

(22) PCT Filed: Jun. 7, 2022
(86) PCT No.: PCT/EP2022/065324

§ 371 Date: Nov. 18, 2024

(51) Int. Cl. C12Q 1/6869; C12N 15/10; C12Q 1/6806; C12Q 1/6855; C12Q 1/686

(52) U.S. Cl. CPC C12Q 1/6869; C12N 15/1096; C12Q 1/6806; C12Q 1/6855; C12Q 1/686; C12Q 2600/16

(57) ABSTRACT

The present invention relates generally to the field of nucleic acid sequencing and provides oligonucleotide molecules and barcodes contained therein. These oligonucleotide molecules and barcodes molecules are useful in sequencing to identify and resolve errors.

### PRIORITY STATEMENT

This application is a national stage application under 35 U.S.C. § 371 of PCT International Application No. PCT/EP2023/065324, which has an international filing date of 7 Jun. 2022. The content of PCT International Application No. PCT/EP2023/065324 is incorporated herein by reference in its entirety.

### REFERENCE TO AN ELECTRONIC SEQUENCE LISTING

This application contains references to nucleic acid sequences and/or amino acid sequences which have been submitted concurrently herewith as the sequence listing .txt file entitled “6170-000178-US-NP_31_Jul_2025_revised_ST25.txt”, file size 147,203 Bytes (B), created on 31 Jul. 2025. The aforementioned sequence listing is hereby incorporated by reference in its entirety.

### FIELD OF THE INVENTION

The present invention relates generally to the field of nucleic acid sequencing and provides oligonucleotide molecules and barcodes contained therein. These oligonucleotide molecules and barcodes molecules are useful in sequencing to identify and resolve errors.

### BACKGROUND OF THE INVENTION

RNA sequencing has become the method of choice for genome-wide transcriptomic analyses as its price has substantially decreased over the last years. Nevertheless, the high cost of standard RNA library preparation and the complexity of the underlying data analysis still prevent this approach from becoming as routine as quantitative PCR (qPCR), especially when many samples need to be analyzed.

To alleviate this high cost, the emerging single-cell transcriptomics field implemented the sample barcoding/early multiplexing principle. This reduces both the RNA-seq cost and preparation time by allowing the generation of a single sequencing library that contains multiple distinct samples/cells (Ziegenhain et al., 2017).

Such a strategy could also be of value to reduce the cost and processing time of bulk RNA sequencing of large sets of samples (Kilpinen et al., 2017; Pradhan et al., 2017; Waszak et al., 2015). However, there have been surprisingly few efforts to explicitly adapt and validate the early-stage multiplexing protocols for reliable and affordable profiling of bulk RNA samples. Early multiplexing protocols designed for single-cell RNA profiling (CEL-seq2, SCRB-seq, and STRT-seq) provide a great capacity for transforming large sets of samples into a unique sequencing library (Hashimshony et al., 2016; Islam et al., 2012; Soumillon et al., 2014). This is achieved by introducing a sample-specific barcode during the RT reaction using a “molecular tag” carried by either the oligo-dT or the template switch oligo (TSO). After individual samples have been “tagged”, they are pooled together, and the remaining steps are performed in bulk, thus shortening the time and cost of library preparation.

Since the tag is introduced to the terminal part of the transcript prior to fragmentation, the reads solely cover the 3′ or 5′ end of the transcripts. The 3′DGE approach for bulk RNA profiling, has been adopted in several recent studies, such as PLATE-seq (Bush et al., 2017), DRUG-seq (Ye et al., 2018), 3′POOL-seq (Sholder et al., 2020), PME-seq (Pandey et al., 2020)) and BRB-seq (Alpern et al., 2019). These techniques have two main commonalities: i) using barcoded DNA oligos used to “tag” poly-adenylated RNA molecules during first strand synthesis and ii) pooling together of all the tagged samples in one tube after the barcoding step.

The overarching goal of these techniques is to decrease the costs and increase the throughput associated with mRNA sequencing library preparation of bulk samples.

This is achieved by reducing reagents, consumables and personnel time through pooling in one solution several barcoded samples. In simple terms, it is much more cost-effective and simpler to process e.g. 96 samples in one tube than 96 samples in 96 tubes.

One of the main challenges of RNA barcoding applied to bulk samples is the ability to guarantee a uniform distribution of sequencing reads across all samples. This challenge is due to the fact that the “molecular barcodes” used during the RNA barcoding step are a functional portion of the “reverse transcription primer” and, as such, different barcodes (i.e. barcodes with different sequences) can have significant effect on the efficiency of the overall workflow. For example, empirical experimental evidence highlights the following potential issues: i) barcodes can lead to unwanted secondary structures that interfere or prevent efficient priming ii) completely random barcodes may end up having very similar or repeating sequences which are then difficult to resolve at the sequencing stage, iii) certain barcodes can be preferentially amplified within the same pool and last, but not least, iv) certain barcodes preferentially bind mitochondrial transcripts, which then appear as an unwanted bias in the sequencing results.

Therefore, there is still a need for more accurate, dependable sequencing tools, (i) and methods that can eliminate barcoding sample-to-sample variation and (ii) methods that use them to improve various barcoding approaches, including the barcoding-mediated high-accuracy sequencing method.

### SUMMARY OF THE INVENTION

The present invention provides one or more oligonucleotide molecules comprising, from 5′ to 3′, a) a sequencing adapter, b) a barcode sequence consisting of 9 to 15 nucleotides, preferably 14 nucleotides, and c) an mRNA capture sequence.

Further provided is set of oligonucleotide molecules, each molecule comprising, from 5′ to 3′, a) a sequencing adaptor, b) a barcode sequence independently selected from the group consisting of SEQ ID: 1 to 96 or a barcode sequence independently selected from the group consisting of SEQ ID: 98 to 481, and c) an mRNA capture sequence.

Further provided is a barcode oligonucleotide sequence selected from the group comprising a sequence as set forth in SEQ ID No. 1 to SEQ ID No. 96, or a combination of one or more thereof.

Further provided is a barcode oligonucleotide sequence selected from the group comprising a sequence as set forth in SEQ ID No. 98 to SEQ ID No. 481, or a combination of one or more thereof.

Further provided is the use of one or more oligonucleotide molecules of the invention, or of a set of oligonucleotide molecules of the invention, or of a barcode oligonucleotide sequence, or a combination of one or more thereof, of the invention, in a sequencing method.

Further provided is a method for providing a cDNA library, the method comprising the steps of
a) Providing a plurality of RNA samples obtained from a biological sample;
b) Contacting separately each RNA sample with one or more oligonucleotide molecules of the invention, or of a library of the invention, or of a barcode oligonucleotide sequence or a combination of one or more thereof, of the invention, under annealing conditions;
c) Incubating separately each sample under reverse transcription reaction conditions;
d) Pooling together all the cDNA:RNA sample;
e) Proceeding to second strand synthesis under synthesis conditions; and
f) Proceeding with tagmentation and amplification under suitable conditions so as to obtain a cDNA library.

Further provided is a method for sequencing RNA, the method comprising the steps of
a) Providing a cDNA library obtained by the method of claims 9 to 10; and
b) Proceeding to the sequencing under suitable conditions.

### DESCRIPTION OF THE FIGURES

FIG. 1 shows the read distribution of the optimal set of 96 barcodes (SEQ ID No. 1 to SEQ ID No. 96) of the invention in which all barcodes are functional and obtain a similar number of sequencing reads.

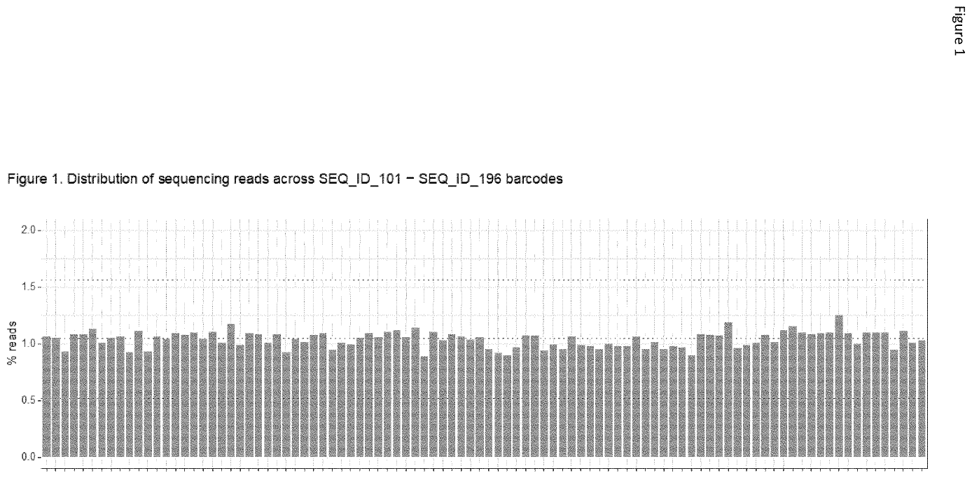

FIG. 2 shows an example of sequencing read distribution for a optimal set of 384 barcodes (SEQ ID No. 98 to SEQ ID No. 481) of the invention in which all barcodes are functional and obtain a similar number of sequencing reads.

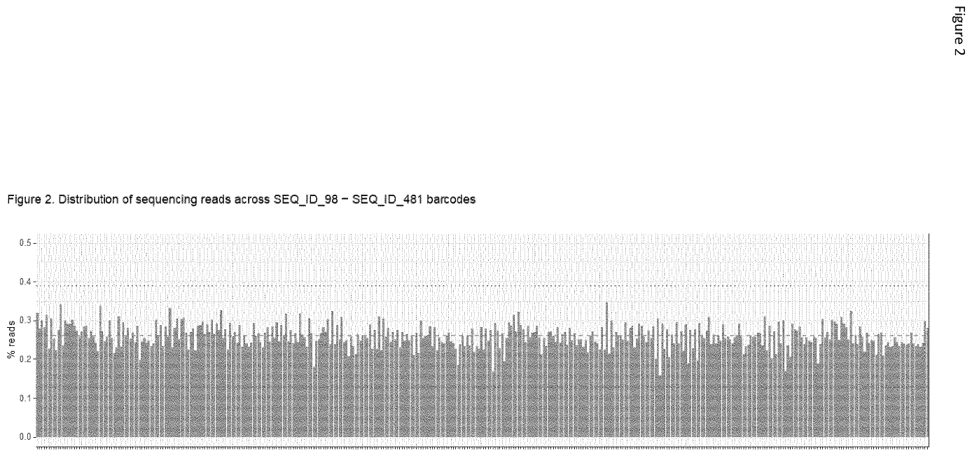

FIG. 3 shows an example of sequencing read distribution for a suboptimal set of 96 barcodes. Barcodes marked with an arrow systematically underperform as compared to the others.

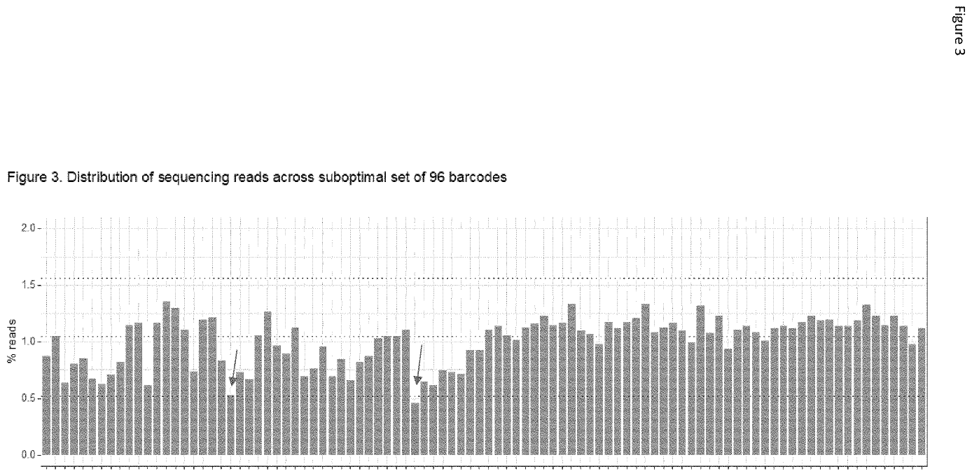

### DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise/comprising” is generally used in the sense of include/including, that is to say permitting the presence of one or more features or components. The terms “comprise(s)” and “comprising” also encompass the more restricted ones “consist(s)”, “consisting” as well as “consist/consisting essentially of”, respectively.

As used in the specification and claims, the singular form “a”, “an” and “the” include plural references unless the context clearly dictates otherwise.

As used herein, “one or more” includes “two or more”, “three or more”, etc. For example, one or more oligonucleotide molecules refers to one oligonucleotide molecule, two oligonucleotide molecules, three oligonucleotide molecules, etc. . . . .

The present invention is based on the discovery of an optimal set barcoded oligonucleotides for multiplexed RNA sequencing. These oligonucleotides contain barcodes that are 14 base pairs long and have been therefore selected from a pool of 4\hat{ }14=268′435′456 potential candidates. This large pool has been filtered twice, first computationally and then experimentally as disclosed herein. The goal was to obtain an optimized set of barcodes for further being able to 1) uniquely demultiplex the samples, with error-tolerance for sequencing errors, and 2) adapt the barcodes for potential technical bias such as overrepresentation of polyT sequences due to preferential amplification of certain sequences.

In one aspect, the invention provides one or more oligonucleotide molecules comprising, from 5′ to 3′, a) a sequencing adapter, b) a barcode sequence consisting of 9 to 15 nucleotides, preferably 14 nucleotides, and, c) an mRNA capture sequence.

In one aspect, the one or more oligonucleotide molecules further comprise d) a unique molecular identifier (UMI). Usually, said UMI consists of 14 nucleotides.

“Oligonucleotide” or “polynucleotide,” which are used synonymously, means a linear polymer of natural or modified nucleosidic monomers linked by phosphodiester bonds or analogs thereof. The term “oligonucleotide” usually refers to a shorter polymer, e.g., comprising from about 3 to about 100 monomers, and the term “polynucleotide” usually refers to longer polymers, e.g., comprising from about 100 monomers to many thousands of monomers, e.g., 10,000 monomers, or more. Oligonucleotides and polynucleotides may be natural or synthetic. Oligonucleotides and polynucleotides include deoxyribonucleosides, ribonucleosides, and non-natural analogs thereof, such as anomeric forms thereof, peptide nucleic acids (PNAs), and the like, provided that they are capable of specifically binding to a target genome by way of a regular pattern of monomer-to-monomer interactions, such as Watson-Crick type of base pairing, base stacking, Hoogsteen or reverse Hoogsteen types of base pairing, or the like.

The terms “peptide,” “protein,” and “polypeptide” are used interchangeably to refer to a natural or synthetic molecule comprising two or more amino acids linked by the carboxyl group of one amino acid to the alpha amino group of another.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

“Sequencing” refers to determining the order of nucleotides (base sequences) in a nucleic acid sample, e.g. DNA or RNA. Many techniques are available such as Sanger sequencing and High Throughput Sequencing technologies (HTS). Sanger sequencing may involve sequencing via detection through (capillary) electrophoresis, in which up to 384 capillaries may be sequence analysed in one run. High throughput sequencing involves the parallel sequencing of thousands or millions or more sequences at once. HTS can be defined as Next Generation sequencing, i.e. techniques based on solid phase pyrosequencing or as Next-Next Generation sequencing based on single nucleotide real time sequencing (SMRT). HTS technologies are available such as offered by Roche, Illumina and Applied Biosystems (Life Technologies). Further high throughput sequencing technologies are described by and/or available from Helicos, Pacific Biosciences, Complete Genomics, Ion Torrent Systems, Oxford Nanopore Technologies, Nabsys, ZS Genetics, GnuBio. Each of these sequencing technologies have their own way of preparing samples prior to the actual sequencing step. Depending on the sequencing technology used, amplification steps may be omitted.

As used herein, the term “barcode” refers to a unique oligonucleotide sequence that allows a corresponding nucleic acid base and/or nucleic acid sequence to be identified. In certain aspects, the nucleic acid base and/or nucleic acid sequence is located at a specific position on a larger polynucleotide sequence (e.g., a polynucleotide covalently attached to a bead). In certain aspects, barcodes can each have a length within a range of from 4 to 150 nucleotides. The barcode technology (or barcoding) has been a particularly powerful technique for studying the genetic and functional variations of the target pool and for high-accuracy target DNA sequencing. Each barcode can comprise deoxyribonucleotides, optionally all of the nucleotides in a barcode region are deoxyribonucleotides. One or more of the deoxyribonucleotides may be a modified deoxyribonucleotide (e.g. a deoxyribonucleotide modified with a biotin moiety or a deoxyuracil nucleotide). The barcodes may comprise one or more degenerate nucleotides or sequences. The barcode regions may not comprise any degenerate nucleotides or sequences.

The barcode sequence of the invention consists of 9 to 15 nucleotides, preferably 14 nucleotides.

In one aspect, the barcode sequence is selected from the group comprising, or consisting of,

SEQ ID NO. 1
TACGTTATTCCGAA
SEQ ID NO. 2
AACAGGATAACTCC
SEQ ID NO. 3
ACTCAGGCACCTCC
SEQ ID NO. 4
ACGAGCAGATGCAG
SEQ ID NO. 5
TTCAATCTCCTTAG
SEQ ID NO. 6
CTCGGTTCGAATGC
SEQ ID NO. 7
TACACTATAGCTAG
SEQ ID NO. 8
CAAGTATAAGGAAC
SEQ ID NO. 9
CTGATATGCAGCGA
SEQ ID NO. 10
ACAATAGGTGGTCC
SEQ ID NO. 11
AACCATCGCATCCA
SEQ ID NO. 12
CAGTCTAGTGCGCA
SEQ ID NO. 13
CCGCAAGAGGTTGG
SEQ ID NO. 14
TAACACCTCCAACG
SEQ ID NO. 15
CAGACATGATAGAG
SEQ ID NO. 16
AACAACCGAAGTAA
SEQ ID NO. 17
AACTAGTATCCGGA
SEQ ID NO. 18
ACGCAGCGAGCAGA
SEQ ID NO. 19
TCCAAGGCATTAAG
SEQ ID NO. 20
TATGACTGCATGCG
SEQ ID NO. 21
ACCGGATAACGGAA
SEQ ID NO. 22
ATAATATTGCCTCC
SEQ ID NO. 23
TCTTGAACGACTAA
SEQ ID NO. 24
TTAACTTACGGTCA
SEQ ID NO. 25
CATGCTAGCGATCG
SEQ ID NO. 26
TAAGCAACAGCCGA
SEQ ID NO. 27
ATGTAGGTAATCGG
SEQ ID NO. 28
CAGCAGCGCTGACA
SEQ ID NO. 29
TATGTACGTAGAGA
SEQ ID NO. 30
CAGATCGCTCTGCG
SEQ ID NO. 31
TAACCTAACTTCAC
SEQ ID NO. 32
CTCCTAACACAGCC
SEQ ID NO. 33
ATACCGTTGGAGGA
SEQ ID NO. 34
CTTATATCGTCGGC
SEQ ID NO. 35
AAGTACTGATCCAA
SEQ ID NO. 36
TACTGACTATCGAG
SEQ ID NO. 37
ATTGCGTCCATTAG
SEQ ID NO. 38
TTGCTACCATTGAA
SEQ ID NO. 39
AAGCGCACCGGAAC
SEQ ID NO. 40
ACCAACTTAGACAA
SEQ ID NO. 41
TCAGCGTGTCGTCA
SEQ ID NO. 42
ATTCCGCTCTGAGG
SEQ ID NO. 43
ACCGATGTGCCGGA
SEQ ID NO. 44
CTAAGGAAGGACCG
SEQ ID NO. 45
TTCTGAGGCGATGG
SEQ ID NO. 46
ACATCTTCTTGAAC
SEQ ID NO. 47
ACAGACGATAGTCA
SEQ ID NO. 48
ACTATCTGCAGTCG
SEQ ID NO. 49
CATGTGTACTAGCA
SEQ ID NO. 50
CTCTGGCTTGCAGG
SEQ ID NO. 51
ACGGCCTTCAACCA
SEQ ID NO. 52
CCATATTGAAGCAC
SEQ ID NO. 53
TCGATTGGTAATGG
SEQ ID NO. 54
TTCATGGTAGAGAG
SEQ ID NO. 55
ACGCTCCTAACGGC
SEQ ID NO. 56
CTCAATTAGAGACC
SEQ ID NO. 57
TCGATCTTCGGCAG
SEQ ID NO. 58
CACATCGACAGCAG
SEQ ID NO. 59
AACCTGCTATCCGG
SEQ ID NO. 60
ATCCGGCGATGACC
SEQ ID NO. 61
TCCATGTGTCCAGG
SEQ ID NO. 62
ATCGATGTCATAGC
SEQ ID NO. 63
CTCCGCATGCTTCC
SEQ ID NO. 64
CACGTAGCTAGACG
SEQ ID NO. 65
AACATGGCACGGAA
SEQ ID NO. 66
ATGCTATCTAGACA
SEQ ID NO. 67
ACCAATCCGTATAA
SEQ ID NO. 68
CTCGTGCCTCAGGA
SEQ ID NO. 69
ATGCAATTACACCG
SEQ ID NO. 70
CCTCCATCCATGGA
SEQ ID NO. 71
ACGGCTCTTAGGCC
SEQ ID NO. 72
TCCAACTACCGAAC
SEQ ID NO. 73
TATGAGAGCTATGA
SEQ ID NO. 74
CAAGGCGTGGTTAC
SEQ ID NO. 75
ATTATCGGATTCGG
SEQ ID NO. 76
ATCCTAAGTGGAGG
SEQ ID NO. 77
ATACGATGCTGCGC
SEQ ID NO. 78
AAGCCTTAGGCCGC
SEQ ID NO. 79
AATGTGTGGATACC
SEQ ID NO. 80
ATGCTGCAACGGCA
SEQ ID NO. 81
CTTGATAAGCCAGG
SEQ ID NO. 82
CATGGAACTCCGGC
SEQ ID NO. 83
TAAGGACCTCATAA
SEQ ID NO. 84
TCTGTCTATGCAGC
SEQ ID NO. 85
CAACCAACCTATAG
SEQ ID NO. 86
CCGGTAGAATGGCC
SEQ ID NO. 87
TCCGAAGTTGTAGA
SEQ ID NO. 88
TCGATGCTATATGA
SEQ ID NO. 89
ACAAGGTTCCGCCG
SEQ ID NO. 90
CCACGTTCCGGTAG
SEQ ID NO. 91
ACTTAAGGTCCAAC
SEQ ID NO. 92
TCCAGAAGGCAACA
SEQ ID NO. 93
CTGCTCATGAGAGA
SEQ ID NO. 94
ATGAATGTTGCCAC
SEQ ID NO. 95
CTCTCAAGTCCTCC
SEQ ID NO. 96
ATTCCAGTTCTTGC,

or a combination of one or more thereof.

Preferably, the combination consists of at least 10 oligonucleotides, at least 20 oligonucleotides, at least 30 oligonucleotides, at least 40 oligonucleotides, at least 50 oligonucleotides, at least 60 oligonucleotides, at least 70 oligonucleotides, at least 80 oligonucleotides, at least 90 oligonucleotides, or a combination of all the oligonucleotides of SEQ ID No. 1 to 96.

In one aspect, the barcode sequence is selected from the group comprising, or consisting of,

SEQ ID NO. 98
TCGTACATAGCGCA
SEQ ID NO. 99
CAAGGAACCGAACC
SEQ ID NO. 100
TCAGTCACGCGTGC
SEQ ID NO. 101
CACACGATACTCAC
SEQ ID NO. 102
AAGGAGAAGAATGC
SEQ ID NO. 103
CAGTGGCCTAAGCA
SEQ ID NO. 104
ACCGTCTTCCTTGC
SEQ ID NO. 105
CCTCATTCGTTGAG
SEQ ID NO. 106
ACGATATGCTCTGC
SEQ ID NO. 107
CCACCGTTAAGACA
SEQ ID NO. 108
ACTACTCGATCTAG
SEQ ID NO. 109
AACACTTGGCCACC
SEQ ID NO. 110
ATCACATCTATCGC
SEQ ID NO. 111
CTCCAATATTCAAC
SEQ ID NO. 112
CCAATAGACTTCCG
SEQ ID NO. 113
ACCGTAACGTGGAC
SEQ ID NO. 114
ATAAGGACCGGAGG
SEQ ID NO. 115
ACCTTCTCACGGCC
SEQ ID NO. 116
CAACGTGGACCTAC
SEQ ID NO. 117
AACTGCTACGCACC
SEQ ID NO. 118
AACTTCCGGCGTGC
SEQ ID NO. 119
CATCGTAGCAGAGC
SEQ ID NO. 120
AACAACCAAGCGGC
SEQ ID NO. 121
TAACCGGCCGCAAC
SEQ ID NO. 122
ATAACCATCCGAAC
SEQ ID NO. 123
CTAGCTGTGTCAGC
SEQ ID NO. 124
CTAACCTTCATTGC
SEQ ID NO. 125
CTCACCTAGGCCAC
SEQ ID NO. 126
TCATGCGCATGTAG
SEQ ID NO. 127
TCGCAATTACCTAA
SEQ ID NO. 128
CATCTACGCTACGC
SEQ ID NO. 129
TTCCACCGATGTGG
SEQ ID NO. 130
TAGCTTCTCCAGGC
SEQ ID NO. 131
TTCCAACCGGACAG
SEQ ID NO. 132
TTGACCTTGCTGGA
SEQ ID NO. 133
ACCTTGTCCAATAC
SEQ ID NO. 134
CTTAGAATTAGTGG
SEQ ID NO. 135
AAGTGACCGACGGC
SEQ ID NO. 136
TCGGTTATCTTCGA
SEQ ID NO. 137
CTAGCTTGTTGTAG
SEQ ID NO. 138
TTAAGCCGAACAAG
SEQ ID NO. 139
TTCCGATGTCCGGC
SEQ ID NO. 140
ATGAATCAGCCGCC
SEQ ID NO. 141
TCCTAACAAGTGCG
SEQ ID NO. 142
CAATAGCAACACCA
SEQ ID NO. 143
TATACGCGACATCG
SEQ ID NO. 144
CCGGATGGTGGTAC
SEQ ID NO. 145
CCTACGGTGTTCGG
SEQ ID NO. 146
CTCGCACCTGGTAA
SEQ ID NO. 147
TCGCCTGAACTACC
SEQ ID NO. 148
TCACTCATACATAG
SEQ ID NO. 149
CTTGGTCTCCGGCA
SEQ ID NO. 150
TTGATTACCGTAAC
SEQ ID NO. 151
TTGATCCGTGAAGC
SEQ ID NO. 152
ATTCTTCAGGAAGA
SEQ ID NO. 153
AAGCCAGGTAAGGC
SEQ ID NO. 154
CCGCGGAAGGATAA
SEQ ID NO. 155
CATAATTCTATGCC
SEQ ID NO. 156
ATGAACTTGGCACA
SEQ ID NO. 157
CTTAAGCTTAAGAG
SEQ ID NO. 158
TCCTCGGAATGACA
SEQ ID NO. 159
AATCTTACCTAAGG
SEQ ID NO. 160
ACCACTGGTTGCCG
SEQ ID NO. 161
CTCCGTTCTGTACC
SEQ ID NO. 162
CCTAACTCGCCAGA
SEQ ID NO. 163
AATAGTGTTCAACC
SEQ ID NO. 164
CTACGAGTCATAGA
SEQ ID NO. 165
ACACACGACATCGC
SEQ ID NO. 166
AACTTACACGTTGG
SEQ ID NO. 167
AACACAACCTTCCG
SEQ ID NO. 168
ATACCTTAATCTCC
SEQ ID NO. 169
TAACCGCAATAGGA
SEQ ID NO. 170
CCGAACCATAGCCG
SEQ ID NO. 171
TTCATATAAGGCGC
SEQ ID NO. 172
AATACCAGCCGCGG
SEQ ID NO. 173
CTACGGACTAAGGC
SEQ ID NO. 174
TAATCGCCTACAGA
SEQ ID NO. 175
TTAGCCTAACTCGC
SEQ ID NO. 176
TAGTGTGGTAGACC
SEQ ID NO. 177
CAACGCCATAGACC
SEQ ID NO. 178
TAATCCATCAACGC
SEQ ID NO. 179
CCGGTGTGCGCTAA
SEQ ID NO. 180
TACCGGTATGGTCC
SEQ ID NO. 181
CACGTATAGATCGC
SEQ ID NO. 182
AACCTAAGCAATCC
SEQ ID NO. 183
CTAGTGTTCGATCC
SEQ ID NO. 184
CCTACACTTGAGCC
SEQ ID NO. 185
CAGCATCATACTGC
SEQ ID NO. 186
AATACGGAGACGGC
SEQ ID NO. 187
ATTGAACTCCATCC
SEQ ID NO. 188
AAGAGTCGGATAGG
SEQ ID NO. 189
AATGCAAGACAACC
SEQ ID NO. 190
TTCATTGGAAGAGA
SEQ ID NO. 191
TTCTAGGACGGCCG
SEQ ID NO. 192
CTCAACCACGTTCC
SEQ ID NO. 193
CTGGTCTGTCTTCC
SEQ ID NO. 194
TTCAAGTCAGTGCC
SEQ ID NO. 195
CACACGACAGAGCG
SEQ ID NO. 196
CCTAAGCCTGTTCG
SEQ ID NO. 197
AAGCTGGCGCCTAG
SEQ ID NO. 198
ACGCCTGTTGGAGG
SEQ ID NO. 199
TCTCCATTATACAG
SEQ ID NO. 200
TCCGCCGCACTCAA
SEQ ID NO. 201
TATCGATCATCTAC
SEQ ID NO. 202
TCTTGTACCGGACC
SEQ ID NO. 203
AACGGCTGAACGAC
SEQ ID NO. 204
TTCCGAAGCCTAAG
SEQ ID NO. 205
TTCTAGATAGGTAC
SEQ ID NO. 206
AATTCTCTGGCGCC
SEQ ID NO. 207
ATGACTGAGAGTGA
SEQ ID NO. 208
TAATTAGGTCCTCG
SEQ ID NO. 209
AACCGTCCAAGCGC
SEQ ID NO. 210
ACATTATTGGCCAA
SEQ ID NO. 211
TCCACAGAACCGGC
SEQ ID NO. 212
TAGTGATCTGTAGC
SEQ ID NO. 213
CATTCACGCTTGAA
SEQ ID NO. 214
CACACACGAGATGC
SEQ ID NO. 215
CCATGACCACCGGA
SEQ ID NO. 216
CAACAAGACCTAAG
SEQ ID NO. 217
AAGGAACCTCTCGG
SEQ ID NO. 218
CATAACCGGCACAA
SEQ ID NO. 219
CCAAGTGGTACAGC
SEQ ID NO. 220
TTCCTTACCGCGCG
SEQ ID NO. 221
ATACTAGTAGTCAG
SEQ ID NO. 222
TATATGCTCGCTCA
SEQ ID NO. 223
TCCGCGATCTCAAC
SEQ ID NO. 224
TATTACCGCTCAGG
SEQ ID NO. 225
ATCGAGCAGTGTAC
SEQ ID NO. 226
CATGCGTAGCGCAC
SEQ ID NO. 227
TCCAACAGTGTTGG
SEQ ID NO. 228
CACTTGGACCATCC
SEQ ID NO. 229
TTACCTGGCCATCC
SEQ ID NO. 230
CATCATGAGAGCGA
SEQ ID NO. 231
ATTCCGAATGTAAC
SEQ ID NO. 232
CCGGTATATCCGGA
SEQ ID NO. 233
ACTGACGTGGAACC
SEQ ID NO. 234
TTAAGCTGCCTTAA
SEQ ID NO. 235
CTCCGCAGTTGGAG
SEQ ID NO. 236
TCCTAATTCTAAGG
SEQ ID NO. 237
TACGATCGTGATCA
SEQ ID NO. 238
ATAACCAACTAGGA
SEQ ID NO. 239
ATCCTTATACGTGC
SEQ ID NO. 240
ACTTCCAGGCCTGA
SEQ ID NO. 241
TCTGCACATCACGC
SEQ ID NO. 242
ACAGACGCATCACG
SEQ ID NO. 243
TAAGTTGTGTGTCC
SEQ ID NO. 244
ACTCAACGGCAAGG
SEQ ID NO. 245
TAGCACATACTCGA
SEQ ID NO. 246
ATATCACAGTCGCA
SEQ ID NO. 247
ATATACACATGAGC
SEQ ID NO. 248
ACGTACGCGCATGC
SEQ ID NO. 249
CAAGCCAGTTCCGG
SEQ ID NO. 250
CCGAACAACCGTGA
SEQ ID NO. 251
CACACACATAGCGA
SEQ ID NO. 252
ACAGCGTACGCGCA
SEQ ID NO. 253
TCAACGCTCGGAGC
SEQ ID NO. 254
ACTACGATCATAGA
SEQ ID NO. 255
CAGGCGTAAGTGCC
SEQ ID NO. 256
ACATATCATGAGCA
SEQ ID NO. 257
AACTCCGTACAAGC
SEQ ID NO. 258
CTGTCGAGTAGCAG
SEQ ID NO. 259
TAGACGATAGTACA
SEQ ID NO. 260
CATGCTCACGCAGC
SEQ ID NO. 261
ATCACGAGCGACGC
SEQ ID NO. 262
CAGCCTGGCTTAGG
SEQ ID NO. 263
CCAGAGGTTGCCAG
SEQ ID NO. 264
TACTATGCACGACG
SEQ ID NO. 265
ACGCTGACGTGCGA
SEQ ID NO. 266
CAGCACATGTACAG
SEQ ID NO. 267
ACACGCATCATACG
SEQ ID NO. 268
CTGTACACGTCTAC
SEQ ID NO. 269
CACACAGTGAGTAC
SEQ ID NO. 270
TAGCACGCGATGAG
SEQ ID NO. 271
TCCAATAGGTCCAG
SEQ ID NO. 272
CTCCTGCGGTCCAA
SEQ ID NO. 273
ATGGCAACGGCTCA
SEQ ID NO. 274
CCGAGGCTTCCTAG
SEQ ID NO. 275
ACGACAGTAGTCGC
SEQ ID NO. 276
TTATAACTGGCAAC
SEQ ID NO. 277
ACTCTGAGTATCAG
SEQ ID NO. 278
TTATGTCTTAACGG
SEQ ID NO. 279
ATGCGACTCGAGAC
SEQ ID NO. 280
CCTCAATTCCGGCG
SEQ ID NO. 281
AATGGTTGGCAGAG
SEQ ID NO. 282
ATGCAAGCGATTCC
SEQ ID NO. 283
TATTGAGTCCGAGA
SEQ ID NO. 284
CCGCGTATTGCAAC
SEQ ID NO. 285
TCATTCTACTTGGC
SEQ ID NO. 286
ATCATCGCGCGACG
SEQ ID NO. 287
CAGAGACACTCGCA
SEQ ID NO. 288
AAGAAGAGTGGACC
SEQ ID NO. 289
ACACACGCTGCGAC
SEQ ID NO. 290
TCCGCAAGTCATAG
SEQ ID NO. 291
AATGTCCTCAGAAC
SEQ ID NO. 292
CCGAGTGTAAGTCC
SEQ ID NO. 293
ACACGTACTCATAC
SEQ ID NO. 294
TTCTCTCCGGTTCA
SEQ ID NO. 295
AATCCGTGTCTTAA
SEQ ID NO. 296
CCTTGCCAGAGTGG
SEQ ID NO. 297
CTTACTGACAAGCC
SEQ ID NO. 298
TCCTCCGATGCCAG
SEQ ID NO. 299
TTCTTCTCCAGAAG
SEQ ID NO. 300
CACCTGTTGGTTAA
SEQ ID NO. 301
CTCAGTCACACGAG
SEQ ID NO. 302
CCTTATCTACGACC
SEQ ID NO. 303
AACCGAACGCTTAA
SEQ ID NO. 304
ACAGGTAGGAAGGA
SEQ ID NO. 305
CAGCATAGTCTGAC
SEQ ID NO. 306
ATTCGGTCCGATGC
SEQ ID NO. 307
ACTAACCTGGCCGG
SEQ ID NO. 308
CATTCCTCTAGCCG
SEQ ID NO. 309
CTTAATCCATATCC
SEQ ID NO. 310
AACAAGAGGTGTGG
SEQ ID NO. 311
TATCATATGAGTCG
SEQ ID NO. 312
ACGAGTATGTATGC
SEQ ID NO. 313
TCGCGGATTAGGCA
SEQ ID NO. 314
CTACTGTGACACAC
SEQ ID NO. 315
CAACCGTGTGAACC
SEQ ID NO. 316
CAGCACTAGCTGCA
SEQ ID NO. 317
ACACGACATGCACG
SEQ ID NO. 318
ACTCATGTCAGACA
SEQ ID NO. 319
ACACTATACACGAG
SEQ ID NO. 320
ACTTGAGCAACCGG
SEQ ID NO. 321
TTAGACGCCGGTAA
SEQ ID NO. 322
CTGTGTAGGATTAA
SEQ ID NO. 323
CACGCATCAGTCAG
SEQ ID NO. 324
CAACCATTGGCAAG
SEQ ID NO. 325
TCATCTGCGCACAC
SEQ ID NO. 326
TAGGCCTCTGATGG
SEQ ID NO. 327
TAGCATGTCAGCAC
SEQ ID NO. 328
TCCTGGACGATGCC
SEQ ID NO. 329
ATATCACATGACAG
SEQ ID NO. 330
ACTACGTCGACACG
SEQ ID NO. 331
AATCCAACTCGAAG
SEQ ID NO. 332
AAGGATGCCAGTGG
SEQ ID NO. 333
ACCGCCTTGGCTAG
SEQ ID NO. 334
CTGTGATTCGGACC
SEQ ID NO. 335
CATGAGACGTCGCG
SEQ ID NO. 336
AATCCGCCTAATGG
SEQ ID NO. 337
AATGCCGTATTGAC
SEQ ID NO. 338
CTCCGAAGAACCGG
SEQ ID NO. 339
ACTGATTAGAATCG
SEQ ID NO. 340
ACTCGCCACCGAAG
SEQ ID NO. 341
CCGGTATTGTTACA
SEQ ID NO. 342
TTGGTGAGAACCGC
SEQ ID NO. 343
CAGATCTCAGACAC
SEQ ID NO. 344
CCTTAACACGGAGA
SEQ ID NO. 345
ATGTCTAGTATAGC
SEQ ID NO. 346
CCAGAGGTAATGGA
SEQ ID NO. 347
CTGTCACACAGTCA
SEQ ID NO. 348
TCACTGAGTGCTGA
SEQ ID NO. 349
TCCTATGGTAGGAG
SEQ ID NO. 350
AAGGAACAATACAC
SEQ ID NO. 351
TAAGCCGGAATTAG
SEQ ID NO. 352
ATAATGCTCCAGAA
SEQ ID NO. 353
ATTCTTGCGAAGCG
SEQ ID NO. 354
ACATGCTATACTAC
SEQ ID NO. 355
AAGCCAACAGTTGG
SEQ ID NO. 356
TATGAGCACACGAC
SEQ ID NO. 357
CACTAGATATCACA
SEQ ID NO. 358
TCAATTAAGGCACG
SEQ ID NO. 359
TCCTCCTCTTGTGA
SEQ ID NO. 360
TACTACTATGTGAC
SEQ ID NO. 361
TATCTTCCACCTGA
SEQ ID NO. 362
CACTTACCTTACAA
SEQ ID NO. 363
TCTAGCATCGACGA
SEQ ID NO. 364
ACACGAGCACGAGC
SEQ ID NO. 365
ACAAGGCAACAGCC
SEQ ID NO. 366
CACTACGCGCTCAC
SEQ ID NO. 367
TAACCGTCTCTGGC
SEQ ID NO. 368
CCAACTCGCCAGGA
SEQ ID NO. 369
CTTCGGTACACTAA
SEQ ID NO. 370
CCACACCTTCAGGC
SEQ ID NO. 371
CCACTTCTTGTTGG
SEQ ID NO. 372
CTAGTCTTGGTAGG
SEQ ID NO. 373
TCAGAATCCTCGCC
SEQ ID NO. 374
CTAGGAGCTTGAAC
SEQ ID NO. 375
CCGGCGCAATTCAG
SEQ ID NO. 376
ACCGGTCGTTCTGC
SEQ ID NO. 377
CATTAAGGTATCGC
SEQ ID NO. 378
CTTGGAGTGGCACA
SEQ ID NO. 379
AACCTTCTAGAGAA
SEQ ID NO. 380
CATGGAATGTGTAA
SEQ ID NO. 381
CTGTTCGGAGGTCA
SEQ ID NO. 382
ATCGTGTGTGGCCA
SEQ ID NO. 383
TTGTCGGTTGAGGC
SEQ ID NO. 384
CTGGCGTTGAGTCG
SEQ ID NO. 385
AATGACCACGACCG
SEQ ID NO. 386
ATGTCTACAGACGA
SEQ ID NO. 387
TCTCCGAGGTAGGC
SEQ ID NO. 388
ACGACGACTGACAG
SEQ ID NO. 389
ACTATGACTGCGCA
SEQ ID NO. 390
CCGCCTTCTAACAC
SEQ ID NO. 391
CACACGCAGTATCA
SEQ ID NO. 392
CCAGCTTAGGAAGA
SEQ ID NO. 393
ACACGATCTGACGA
SEQ ID NO. 394
AAGGCAAGTTGTGA
SEQ ID NO. 395
AACTCTTCGGTAGG
SEQ ID NO. 396
CCGTCCTTCTTCAC
SEQ ID NO. 397
CTTGGTTGGTTCCG
SEQ ID NO. 398
TACCTTGGAGTTGG
SEQ ID NO. 399
ACCGGCGGTATAGG
SEQ ID NO. 400
CACGACGACTCAGA
SEQ ID NO. 401
TCTTGTGAGGTTCG
SEQ ID NO. 402
AACGCTTCCGGTCC
SEQ ID NO. 403
CTACTGCATAGTAG
SEQ ID NO. 404
ACACAGCACGATAG
SEQ ID NO. 405
CCTACAGGTCGAGG
SEQ ID NO. 406
ATGCACGATCGAGC
SEQ ID NO. 407
CTTGATACCGGCGC
SEQ ID NO. 408
CCTCGGTTGAAGCC
SEQ ID NO. 409
CAACACCGGCTTGG
SEQ ID NO. 410
CTCTGGACCGATCA
SEQ ID NO. 411
CCGTGAACCTTGCG
SEQ ID NO. 412
TCATGCTCGAGACA
SEQ ID NO. 413
ACCGGAACCTATGG
SEQ ID NO. 414
TTCCAAGGTTGACA
SEQ ID NO. 415
ACGAGAGTGCTGAG
SEQ ID NO. 416
CTCAACCGGACGGA
SEQ ID NO. 417
AATCAAGTGTGAAC
SEQ ID NO. 418
CATACGGCCTCTAA
SEQ ID NO. 419
ACTAGAGCGTGTGA
SEQ ID NO. 420
TCAGAGTAGTGCAG
SEQ ID NO. 421
CAACAACCTTGAGG
SEQ ID NO. 422
ACAATTAGCCTTGG
SEQ ID NO. 423
TCACTATGCGATAC
SEQ ID NO. 424
TACTCTCATCTAGC
SEQ ID NO. 425
TCTCTCTCAATTGG
SEQ ID NO. 426
CCAGTGAGGTATAC
SEQ ID NO. 427
CTCCTACTTAATGC
SEQ ID NO. 428
ATAGGCCTTCGAGG
SEQ ID NO. 429
CATTGGTGGTGTCC
SEQ ID NO. 430
CATACTATGTAGAC
SEQ ID NO. 431
ATGGATCGTTAGGA
SEQ ID NO. 432
CCAACCGAACGGCA
SEQ ID NO. 433
ACGTACATCTATCG
SEQ ID NO. 434
TACGAGTGTCTCAG
SEQ ID NO. 435
ATAACAACGAAGCC
SEQ ID NO. 436
TACGATAGTACAGC
SEQ ID NO. 437
ATTGCGGTTAATCA
SEQ ID NO. 438
CAGTTGCGAAGTGG
SEQ ID NO. 439
TTAACACCTCAAGG
SEQ ID NO. 440
CACGACATGCGAGC
SEQ ID NO. 441
TCCAGAACTGAGGC
SEQ ID NO. 442
TATTCCTAGTTCGA
SEQ ID NO. 443
AACACCTTCTCCGC
SEQ ID NO. 444
TAACCTGTTCAAGA
SEQ ID NO. 445
AAGCTATGTGCAAC
SEQ ID NO. 446
CTCTTAATCCTTGA
SEQ ID NO. 447
ATCAGCAGACTAGC
SEQ ID NO. 448
CAAGGAAGCATTGG
SEQ ID NO. 449
ACCTCGAAGTTCAA
SEQ ID NO. 450
ATAGCACTCACACG
SEQ ID NO. 451
ATCTAGCTGCACGA
SEQ ID NO. 452
TCTGCTGCAGAGCG
SEQ ID NO. 453
TAACAGTCCGTTCA
SEQ ID NO. 454
CATCTATCTGCGCG
SEQ ID NO. 455
TTGACCAAGATTAC
SEQ ID NO. 456
TCTCACATCACGAG
SEQ ID NO. 457
ACACATCTCTACGA
SEQ ID NO. 458
AACAGAATAGGCGG
SEQ ID NO. 459
AACACCTAAGGAAG
SEQ ID NO. 460
AACACCTGGTTGAA
SEQ ID NO. 461
AAGACGAATAGGAA
SEQ ID NO. 462
CAAGTTCAGAATGG
SEQ ID NO. 463
CCAAGCTTAGTGCG
SEQ ID NO. 464
AACGCTCAATGTGG
SEQ ID NO. 465
TCATCTAGGTGGAA
SEQ ID NO. 466
CCTGTGATTACACC
SEQ ID NO. 467
TCAGAACAGGTTAA
SEQ ID NO. 468
ATATTGTAAGGTGG
SEQ ID NO. 469
TTAAGTGACCGCGC
SEQ ID NO. 470
AATCCTGAATCCAA
SEQ ID NO. 471
CTATAGCGCACTAC
SEQ ID NO. 472
TCTAGACACTATCG
SEQ ID NO. 473
TCAATGGCAGGCCG
SEQ ID NO. 474
AAGTGGTATTGACA
SEQ ID NO. 475
TTGGACATTCCGGC
SEQ ID NO. 476
CAGCATCGAGAGCG
SEQ ID NO. 477
CAAGACCTTGGCCA
SEQ ID NO. 478
CCGGTTCCTAGTGA
SEQ ID NO. 479
ACAACTTAGCCTAA
SEQ ID NO. 480
TCGTGTCGCATGCA
SEQ ID NO. 481
CACACAGACGCTCG,

or a combination of one or more thereof.

Preferably, the combination consists of at least 10 oligonucleotides, at least 20 oligonucleotides, at least 30 oligonucleotides, at least 40 oligonucleotides, at least 50 oligonucleotides, at least 60 oligonucleotides, at least 70 oligonucleotides, at least 80 oligonucleotides, at least 90 oligonucleotides, at least 100 oligonucleotides, at least 150 oligonucleotides, at least 200 oligonucleotides, at least 250 oligonucleotides, at least 300 oligonucleotides, or a combination of all 384 oligonucleotides of SEQ ID No. 98 to 481.

As used herein, the term “sequencing adapter” refers to an oligonucleotide sequence that can be used in subsequent sequencing steps (so-called sequencing adapters), or to primers that are used to amplify a subset of fragments prior to sequencing may contain parts within their sequence that introduce sections that can later be used in the sequencing step, for instance by introducing through an amplification step a sequencing adapter or a capturing moiety in an amplicon that can be used in a subsequent sequencing step. Depending also on the sequencing technology used, amplification steps may be omitted.

Any commercially available sequencing adapter can be used. In one aspect, the sequencing adapter comprises, or consists of, SEQ ID No. 97 (CTA CAC GAC GCT CTT CCG ATC T).

As used herein, a “unique molecular identifier” or UMI is a complex indices added to sequencing libraries before any PCR amplification steps, enabling the accurate bioinformatic identification of PCR duplicates thus enabling to remove PCR duplicates. In one aspect, the UMI is an oligonucleotide sequence consisting of a sequence (N)n(V)m, wherein N is any nucleotide selected from A, T, C and G; V is any nucleotide selected from A, C and G; n is an integer selected from 1 to 20, and m is an integer selected from 1 to 20. In a preferred aspect, the UMI is an oligonucleotide sequence consisting of NNNNNNNNNVVVVV (SEQ ID No. 482).

As used herein, an “mRNA capture sequence” is an oligonucleotide sequence that specifically hybridizes to mRNAs. In one aspect, the mRNA capture sequence is a poly-T sequence. In a preferred aspect, the mRNA capture sequence is a poly-T sequence followed by at least one V and one N, wherein N is any nucleotide selected from A, T, C and G and V is any nucleotide selected from A, C and G. Example of an mRNA capture sequence consists of TTTTTTTTTTTTTTTTTTTTTTTTTTTTTTVN (SEQ ID No. 483).

“Multiplex sequencing” refers to a sequencing technique that allows for processing a large number of samples on a high-throughput instrument. For multiplex sequencing, individual “barcode” sequences of the invention are added to each sample so that nucleotide sequences from different samples can be distinguished by the unique barcode sequences embedded in each sample. With this technique, multiple DNA or RNA samples can be pooled, processed, sequenced, and analyzed simultaneously.

The present invention further provides a set of oligonucleotide molecules, each molecule comprising, from 5′ to 3′,
a) a sequencing adaptor,
b) a barcode sequence independently selected from the group consisting of SEQ ID: 1 to 96 or a barcode sequence independently selected from the group consisting of SEQ ID: 98 to 481, and
c) an mRNA capture sequence.

In one aspect, the set of oligonucleotide molecules of the invention, further comprises d) a UMI as described herein.

Also provided is the use of one or more oligonucleotide molecules of the invention, or of a set of oligonucleotide molecules of the invention, or of a barcode oligonucleotide sequence, or a combination of one or more thereof, of the invention, in a sequencing method.

The one or more oligonucleotide molecules of the invention, or of a set of oligonucleotide molecules of the invention, or of a barcode oligonucleotide sequence, or a combination of one or more thereof, may be linked by attachment to a solid support (e.g. a bead). A solution of soluble beads (e.g. superparamagnetic beads or styrofoam beads) may be functionalized to enable attachment of two or more oligonucleotide molecules of the invention, set of oligonucleotide molecules of the invention, barcode oligonucleotide sequences, or a combination of one or more thereof. This functionalization may be enabled through chemical moieties (e.g. carboxylated groups), and/or protein-based adapters (e.g. streptavidin) on the beads. The functionalized beads may be brought into contact with a solution of the above-described molecules under conditions which promote the attachment of two or more molecules to each bead in the solution. Optionally, the molecules are attached through a covalent linkage, or through a (stable) non-covalent linkage such as a streptavidin-biotin bond, or a (stable) oligonucleotide hybridization bond.

The present invention further encompasses a method for providing a cDNA library, the method comprising the step of
Providing a plurality of RNA samples obtained from a biological sample (step a).

As used herein, the term “biological sample” refers to a tissue (e.g., tissue biopsy), organ, cell (including a cell maintained in culture), cell lysate (or lysate fraction), biomolecule derived from a cell or cellular material (e.g. a polypeptide or nucleic acid), or body fluid from a subject. Non-limiting examples of body fluids include blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid.

The RNA samples can be obtained from any techniques know in the art. According to a particular aspect, the RNA samples are mRNA samples that can be cell lysates, total DNA/RNA eluate, blood and FFPE tissues.

The method further comprises a step (b) of contacting separately each RNA sample with one or more oligonucleotide molecules of the invention, or of a library of the invention, or of a barcode oligonucleotide sequence or a combination of one or more thereof, of the invention, under annealing conditions.

For examples, RNA samples are thawed on ice, transferred to the corresponding wells of the Oligo-dT primer plat, the plate is then sealed with the AluSeal and placed it in a thermocycler at 65° C. for 5 min and immediately put on ice.

The method further comprises a step (c) of incubating separately each sample under reverse transcription reaction conditions.

For example, the RT reaction mix is prepared according to commercial manufacture instruction. Any RT enzyme and buffer commercially available can be used, such as e.g. Lucigen's ERT12910K, ThermoFisher's 18064014 and NEB's M0368S among others.

For example.

| RT Mix | Per well (μL) |
|---|---|
| RT reaction buffer | 5.0 |
| RT reaction enzyme | 0.4 |
| ddH2O | 4.6 |
| TOTAL | 10.0 |

Incubate RT reaction mix in thermocycler with the following program:

| Step | Temperature, ° C. | Time |
|---|---|---|
| Incubation | 42 | 50 min |
| Inactivation | 70 | 10 min |
| Keep | 4 | pause |

The method further comprises a step (d) of pooling together all the cDNA:RNA sample. The method further comprises a step (e) of proceeding to second strand synthesis under synthesis conditions such as. This second strand synthesis can be generated by any method known in the art. In one aspect, the second strand synthesis method is selected from the group comprising PCR amplification and nick translation, or a combination thereof.

The method further comprises a step (f) of proceeding with tagmentation, and/or end-repair and ligation and amplification under suitable conditions such as, e.g. the conditions described in the examples, so as to obtain a cDNA library.

Examples of RNAs include but are not limited to: mRNA, amplicons, rRNA, tRNA, nRNA, siRNA, snRNA, snoRNA, scaRNA, microRNA, dsRNA, ncRNA (e.g. lncRNA), ribozyme, riboswitch and viral RNA (e.g., retroviral RNA).

Examples of RNAs include but are not limited to: mRNA, amplicons, rRNA, tRNA, nRNA, siRNA, snRNA, snoRNA, scaRNA, microRNA, dsRNA, ncRNA (e.g. lncRNA), ribozyme, riboswitch and viral RNA (e.g., retroviral RNA).

The present invention further encompasses a method for sequencing RNA, the method comprising the steps of
a) Providing a cDNA library obtained by the method described herein; and
b) Proceeding to the sequencing under suitable conditions such as, e.g. those defined by NGS sequencing providers, which are also known in the art.

In one aspect, the one or more oligonucleotide molecules of the invention may be linked by attachment to a solid support (e.g. a bead).

Also contemplated is one or more kits for performing one or more methods according to the invention. The one or more kits comprising
i) one or more oligonucleotide molecules of any one of the invention, or of a set of oligonucleotide molecules of the invention, or of a barcode oligonucleotide sequence, or a combination of one or more thereof of the invention,
ii) a support for sample preparation, such as a 96-well plate or 384-well plate or a set of 96-well plates combining 384 oligos with different primers, and
iii) reagents for sequencing.

The kit(s) can comprise various molecular biology reagents, including DNA polymerases, RNA polymerases, reverse-transcriptases, DNA ligases, RNA ligases, transposases, viral integrase, CRISPR/Cas9, zinc finger nucleases, transcription activator-like effector nucleases, exonucleases, endonucleases, polynucleotide kinases, nucleotides, oligonucleotides, modified oligonucleotides, optimized buffers and cell lysis reagents.

Further contemplated is the use of the kit(s) of the invention, or of the one or more oligonucleotide molecules of the invention, or of a library of the invention, or of a barcode oligonucleotide sequence or a combination of one or more thereof, of the invention, in a single-cell RNA profiling method. Any single cell RNA profiling known in the art are considered. Preferably, the single-cell RNA profiling method is the Bulk RNA Barcoding and sequencing (BRB-seq) method described in (Alpern et al., 2019) or an adaptation thereof.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein. Various references are cited throughout this Specification, each of which is incorporated herein by reference in its entirety. The foregoing description will be more fully understood with reference to the following Examples.

### Examples

### Second-Strand Synthesis

Double-stranded cDNA was generated by nick translation (Gubler and Hoffman, 1983). For that, a mix containing 2 μL of RNAse H (NEB, #M0297S), 1 μL of Escherichia coli DNA ligase (NEB, #M0205 L), 5 μL of E. coli DNA Polymerase (NEB, #M0209 L), 1 μL of dNTP (0.2 mM), 10 μL of 5× Second Stand Buffer (100 mM Tris-HCl (pH 6.9) (AppliChem, #A3452); 25 mM MgCl2 (Sigma, #M2670); 450 mM KCl (AppliChem, #A2939); 0.8 mM (3-NAD; 60 mM (NH4)2SO4 (Fisher Scientific Acros, #AC20587); and 11 μL of water was added to 20 μL of ExoI-treated first-strand reaction on ice. The reaction was incubated at 16° C. for 2.5 h or overnight. Full-length double-stranded cDNA was purified with 30 μL (0.6×) of AMPure XP magnetic beads (Beckman Coulter, #A63881) and eluted in 20 μL of water. Alternatively, double stranded cDNA was generated by PCR amplification following template switch oligo assisted first strand synthesis. PCR amplification was performed in 50 μL reaction using NEBNext High-Fidelity 2×PCR Master Mix (NEB, #M0541 L) and 1 μL of primers CTACACGACGCTCTTCCGATCT (SEQ ID No. 484) and AAGCAGTGGTATCAACGCAGAG (SEQ ID No. 485) (10 μM, IDT).

### Library Preparation and Sequencing

The sequencing libraries were prepared by tagmentation of 1-50 ng of full-length double-stranded cDNA. Tagmentation was done either with Illumina Nextera XT kit (Illumina, #FC-131-1024) following the manufacturer's recommendations or with in-house produced Tn5 preloaded with dual (Tn5-A/B) or same adapters (Tn5-B/B) under the following conditions: 1 μL (11 μM) Tn5, 4 μL of 5×TAPS buffer (50 mM TAPS (Sigma, #T5130), and 25 mM MgCl2 (Sigma, #M2670)) in 20 μL total volume. The reaction was incubated 10 min at 55° C. followed by purification with DNA Clean & Concentrator-5 kit (Zymo Research) and elution in 21 μL of water. After that, tagmented library (20 μL) was PCR amplified using 25 μL NEBNext High-Fidelity 2×PCR Master Mix (NEB, #M0541 L), 2.5 μL of each of P5 and P7 indexing primers bearing library indexing sequence (5 M, IDT), using the following program: incubation 72° C.-3 min, denaturation 98° C.-30 s; 10 cycles: 98° C.-10 s, 63° C.-30 s, 72° C.-30 s; final elongation at 72° C.-5 min. The fragments ranging 200-1000 bp were size-selected using AMPure beads (Beckman Coulter, #A63881) (first round 0.5× beads, second 0.7×). The libraries were profiled with High Sensitivity NGS Fragment Analysis Kit (Agilent, #DNF-474) and measured with Qubit dsDNA HS Assay Kit (Invitrogen, #Q32851) prior to pooling and sequencing using the Illumina instruments (Nextseq, Novaseq, Miseq, iSeq or Hiseq4000). The read1 sequencing was performed for 28 cycles and read2 for 60-90 cycles depending on the experiment.

### REFERENCES

Alpern, D., Gardeux, V., Russeil, J., Mangeat, B., Meireles-Filho, A. C. A., Breysse, R., Hacker, D., and Deplancke, B. (2019). BRB-seq: ultra-affordable high-throughput transcriptomics enabled by bulk RNA barcoding and sequencing. Genome Biol. 20, 71. https://doi.org/10.1186/s13059-019-1671-x.
Bush, E. C., Ray, F., Alvarez, M. J., Realubit, R., Li, H., Karan, C., Califano, A., and Sims, P. A. (2017). PLATE-Seq for genome-wide regulatory network analysis of high-throughput screens. Nat. Commun. 8. https://doi.org/10.1038/s41467-017-00136-z.
Gubler, U., and Hoffman, B. J. (1983). A simple and very efficient method for generating cDNA libraries. Gene 25, 263-269. https://doi.org/10.1016/0378-1119(83)90230-5.
Hashimshony, T., Senderovich, N., Avital, G., Klochendler, A., de Leeuw, Y., Anavy, L., Gennert, D., Li, S., Livak, K. J., Orit, R.-R., et al. (2016). CEL-Seq2: sensitive highly-multiplexed single-cell RNA-Seq. Genome Biol 17, 77. https://doi.org/10.1186/si3059-016-0938-8.
Islam, S., Kjallquist, U., Moliner, A., Zajac, P., Fan, J.-B. B., Lonnerberg, P., and Linnarsson, S. (2012). Highly multiplexed and strand-specific single-cell RNA 5′ end sequencing. Nat Protoc 7, 813-828. https://doi.org/10.1038/nprot.2012.022.
Kilpinen, H., Goncalves, A., Leha, A., Afzal, V., Alasoo, K., Ashford, S., Bala, S., Bensaddek, D., Casale, F. P., Culley, O. J., et al. (2017). Common genetic variation drives molecular heterogeneity in human iPSCs. Nature 546, 370-375. https://doi.org/10.1038/nature22403.
Pandey, S., Takahama, M., Gruenbaum, A., Zewde, M., Cheronis, K., and Chevrier, N. (2020). A whole-tissue RNA-seq toolkit for organism-wide studies of gene expression with PME-seq. Nat. Protoc. https://doi.org/10.1038/s41596-019-0291-y.
Pradhan, R. N., Bues, J. J., Gardeux, V., Schwalie, P. C., Alpern, D., Chen, W., Russeil, J., Raghav, S. K., and Deplancke, B. (2017). Dissecting the brown adipogenic regulatory network using integrative genomics. Sci. Rep. 7, 42130. https://doi.org/10.1038/srep42130.
Sholder, G., Lanz, T. A., Moccia, R., Quan, J., Aparicio-Prat, E., Stanton, R., and Xi, H. S. (2020). 3′Pool-seq: an optimized cost-efficient and scalable method of whole-transcriptome gene expression profiling. BMC Genomics 21. https://doi.org/10.1186/si2864-020-6478-3.
Soumillon, M., Cacchiarelli, D., and Semrau, S. (2014). Characterization of directed differentiation by high-throughput single-cell RNA-Seq. BioRxiv.
Waszak, S. M., Delaneau, O., Gschwind, A. R., Kilpinen, H., Raghav, S. K., Witwicki, R. M., Orioli, A., Wiederkehr, M., Panousis, N. I., Yurovsky, A., et al. (2015). Population Variation and Genetic Control of Modular Chromatin Architecture in Humans. Cell 162, 1039-1050. https://doi.org/10.1016/j.cell.2015.08.001.
Ye, C., Ho, D. J., Neri, M., Yang, C., Kulkarni, T., Randhawa, R., Henault, M., Mostacci, N., Farmer, P., Renner, S., et al. (2018). DRUG-seq for miniaturized high-throughput transcriptome profiling in drug discovery. Nat. Commun. 9. https://doi.org/10.1038/s41467-018-06500-x.
Ziegenhain, C., Vieth, B., Parekh, S., Reinius, B., Guillaumet-Adkins, A., Smets, M., Leonhardt, H., Heyn, H., Hellmann, I., and Enard, W. (2017). Comparative Analysis of Single-Cell RNA Sequencing Methods. Mol. Cell 65, 631-643.e4. https://doi.org/10.1016/j.molcel.2017.01.023.

1. One or more oligonucleotide molecules comprising, from 5′ to 3′,
a) a sequencing adapter,
b) a barcode sequence consisting of 9 to 15 nucleotides, and
c) an mRNA capture sequence.

2. The one or more oligonucleotide molecules of claim 1, further comprising d) a unique molecular identifier (UMI).

3. The one or more oligonucleotide molecules of claim 2, wherein the UMI consists of 14 nucleotides.

4. The one or more oligonucleotide molecules of claim 1, wherein the sequencing adapter comprises CTA CAC GAC GCT CTT CCG ATC T (SEQ ID NO. 97).

5. The one or more oligonucleotide molecules of claim 1, wherein the barcode sequence is selected from the group consisting of:
SEQ ID NO. 1
TACGTTATTCCGAA
SEQ ID NO. 2
AACAGGATAACTCC
SEQ ID NO. 3
ACTCAGGCACCTCC
SEQ ID NO. 4
ACGAGCAGATGCAG
SEQ ID NO. 5
TTCAATCTCCTTAG
SEQ ID NO. 6
CTCGGTTCGAATGC
SEQ ID NO. 7
TACACTATAGCTAG
SEQ ID NO. 8
CAAGTATAAGGAAC
SEQ ID NO. 9
CTGATATGCAGCGA
SEQ ID NO. 10
ACAATAGGTGGTCC
SEQ ID NO. 11
AACCATCGCATCCA
SEQ ID NO. 12
CAGTCTAGTGCGCA
SEQ ID NO. 13
CCGCAAGAGGTTGG
SEQ ID NO. 14
TAACACCTCCAACG
SEQ ID NO. 15
CAGACATGATAGAG
SEQ ID NO. 16
AACAACCGAAGTAA
SEQ ID NO. 17
AACTAGTATCCGGA
SEQ ID NO. 18
ACGCAGCGAGCAGA
SEQ ID NO. 19
TCCAAGGCATTAAG
SEQ ID NO. 20
TATGACTGCATGCG
SEQ ID NO. 21
ACCGGATAACGGAA
SEQ ID NO. 22
ATAATATTGCCTCC
SEQ ID NO. 23
TCTTGAACGACTAA
SEQ ID NO. 24
TTAACTTACGGTCA
SEQ ID NO. 25
CATGCTAGCGATCG
SEQ ID NO. 26
TAAGCAACAGCCGA
SEQ ID NO. 27
ATGTAGGTAATCGG
SEQ ID NO. 28
CAGCAGCGCTGACA
SEQ ID NO. 29
TATGTACGTAGAGA
SEQ ID NO. 30
CAGATCGCTCTGCG
SEQ ID NO. 31
TAACCTAACTTCAC
SEQ ID NO. 32
CTCCTAACACAGCC
SEQ ID NO. 33
ATACCGTTGGAGGA
SEQ ID NO. 34
CTTATATCGTCGGC
SEQ ID NO. 35
AAGTACTGATCCAA
SEQ ID NO. 36
TACTGACTATCGAG
SEQ ID NO. 37
ATTGCGTCCATTAG
SEQ ID NO. 38
TTGCTACCATTGAA
SEQ ID NO. 39
AAGCGCACCGGAAC
SEQ ID NO. 40
ACCAACTTAGACAA
SEQ ID NO. 41
TCAGCGTGTCGTCA
SEQ ID NO. 42
ATTCCGCTCTGAGG
SEQ ID NO. 43
ACCGATGTGCCGGA
SEQ ID NO. 44
CTAAGGAAGGACCG
SEQ ID NO. 45
TTCTGAGGCGATGG
SEQ ID NO. 46
ACATCTTCTTGAAC
SEQ ID NO. 47
ACAGACGATAGTCA
SEQ ID NO. 48
ACTATCTGCAGTCG
SEQ ID NO. 49
CATGTGTACTAGCA
SEQ ID NO. 50
CTCTGGCTTGCAGG
SEQ ID NO. 51
ACGGCCTTCAACCA
SEQ ID NO. 52
CCATATTGAAGCAC
SEQ ID NO. 53
TCGATTGGTAATGG
SEQ ID NO. 54
TTCATGGTAGAGAG
SEQ ID NO. 55
ACGCTCCTAACGGC
SEQ ID NO. 56
CTCAATTAGAGACC
SEQ ID NO. 57
TCGATCTTCGGCAG
SEQ ID NO. 58
CACATCGACAGCAG
SEQ ID NO. 59
AACCTGCTATCCGG
SEQ ID NO. 60
ATCCGGCGATGACC
SEQ ID NO. 61
TCCATGTGTCCAGG
SEQ ID NO. 62
ATCGATGTCATAGC
SEQ ID NO. 63
CTCCGCATGCTTCC
SEQ ID NO. 64
CACGTAGCTAGACG
SEQ ID NO. 65
AACATGGCACGGAA
SEQ ID NO. 66
ATGCTATCTAGACA
SEQ ID NO. 67
ACCAATCCGTATAA
SEQ ID NO. 68
CTCGTGCCTCAGGA
SEQ ID NO. 69
ATGCAATTACACCG
SEQ ID NO. 70
CCTCCATCCATGGA
SEQ ID NO. 71
ACGGCTCTTAGGCC
SEQ ID NO. 72
TCCAACTACCGAAC
SEQ ID NO. 73
TATGAGAGCTATGA
SEQ ID NO. 74
CAAGGCGTGGTTAC
SEQ ID NO. 75
ATTATCGGATTCGG
SEQ ID NO. 76
ATCCTAAGTGGAGG
SEQ ID NO. 77
ATACGATGCTGCGC
SEQ ID NO. 78
AAGCCTTAGGCCGC
SEQ ID NO. 79
AATGTGTGGATACC
SEQ ID NO. 80
ATGCTGCAACGGCA
SEQ ID NO. 81
CTTGATAAGCCAGG
SEQ ID NO. 82
CATGGAACTCCGGC
SEQ ID NO. 83
TAAGGACCTCATAA
SEQ ID NO. 84
TCTGTCTATGCAGC
SEQ ID NO. 85
CAACCAACCTATAG
SEQ ID NO. 86
CCGGTAGAATGGCC
SEQ ID NO. 87
TCCGAAGTTGTAGA
SEQ ID NO. 88
TCGATGCTATATGA
SEQ ID NO. 89
ACAAGGTTCCGCCG
SEQ ID NO. 90
CCACGTTCCGGTAG
SEQ ID NO. 91
ACTTAAGGTCCAAC
SEQ ID NO. 92
TCCAGAAGGCAACA
SEQ ID NO. 93
CTGCTCATGAGAGA
SEQ ID NO. 94
ATGAATGTTGCCAC
SEQ ID NO. 95
CTCTCAAGTCCTCC
SEQ ID NO. 96
ATTCCAGTTCTTGC,
and a combination of one or more thereof.

6. The one or more oligonucleotide molecules of claim 1, consisting of at least 10 oligonucleotides molecules, at least 20 oligonucleotides molecules, at least 30 oligonucleotides molecules, at least 40 oligonucleotides molecules, at least 50 oligonucleotides molecules, at least 60 oligonucleotides molecules, at least 70 oligonucleotides molecules, at least 80 oligonucleotides molecules, at least 90 oligonucleotides molecules, or 96 oligonucleotides molecules.

7. The one or more oligonucleotide molecules of claim 1, wherein the barcode sequence is selected from the group consisting of:
SEQ ID NO. 98
TCGTACATAGCGCA
SEQ ID NO. 99
CAAGGAACCGAACC
SEQ ID NO. 100
TCAGTCACGCGTGC
SEQ ID NO. 101
CACACGATACTCAC
SEQ ID NO. 102
AAGGAGAAGAATGC
SEQ ID NO. 103
CAGTGGCCTAAGCA
SEQ ID NO. 104
ACCGTCTTCCTTGC
SEQ ID NO. 105
CCTCATTCGTTGAG
SEQ ID NO. 106
ACGATATGCTCTGC
SEQ ID NO. 107
CCACCGTTAAGACA
SEQ ID NO. 108
ACTACTCGATCTAG
SEQ ID NO. 109
AACACTTGGCCACC
SEQ ID NO. 110
ATCACATCTATCGC
SEQ ID NO. 111
CTCCAATATTCAAC
SEQ ID NO. 112
CCAATAGACTTCCG
SEQ ID NO. 113
ACCGTAACGTGGAC
SEQ ID NO. 114
ATAAGGACCGGAGG
SEQ ID NO. 115
ACCTTCTCACGGCC
SEQ ID NO. 116
CAACGTGGACCTAC
SEQ ID NO. 117
AACTGCTACGCACC
SEQ ID NO. 118
AACTTCCGGCGTGC
SEQ ID NO. 119
CATCGTAGCAGAGC
SEQ ID NO. 120
AACAACCAAGCGGC
SEQ ID NO. 121
TAACCGGCCGCAAC
SEQ ID NO. 122
ATAACCATCCGAAC
SEQ ID NO. 123
CTAGCTGTGTCAGC
SEQ ID NO. 124
CTAACCTTCATTGC
SEQ ID NO. 125
CTCACCTAGGCCAC
SEQ ID NO. 126
TCATGCGCATGTAG
SEQ ID NO. 127
TCGCAATTACCTAA
SEQ ID NO. 128
CATCTACGCTACGC
SEQ ID NO. 129
TTCCACCGATGTGG
SEQ ID NO. 130
TAGCTTCTCCAGGC
SEQ ID NO. 131
TTCCAACCGGACAG
SEQ ID NO. 132
TTGACCTTGCTGGA
SEQ ID NO. 133
ACCTTGTCCAATAC
SEQ ID NO. 134
CTTAGAATTAGTGG
SEQ ID NO. 135
AAGTGACCGACGGC
SEQ ID NO. 136
TCGGTTATCTTCGA
SEQ ID NO. 137
CTAGCTTGTTGTAG
SEQ ID NO. 138
TTAAGCCGAACAAG
SEQ ID NO. 139
TTCCGATGTCCGGC
SEQ ID NO. 140
ATGAATCAGCCGCC
SEQ ID NO. 141
TCCTAACAAGTGCG
SEQ ID NO. 142
CAATAGCAACACCA
SEQ ID NO. 143
TATACGCGACATCG
SEQ ID NO. 144
CCGGATGGTGGTAC
SEQ ID NO. 145
CCTACGGTGTTCGG
SEQ ID NO. 146
CTCGCACCTGGTAA
SEQ ID NO. 147
TCGCCTGAACTACC
SEQ ID NO. 148
TCACTCATACATAG
SEQ ID NO. 149
CTTGGTCTCCGGCA
SEQ ID NO. 150
TTGATTACCGTAAC
SEQ ID NO. 151
TTGATCCGTGAAGC
SEQ ID NO. 152
ATTCTTCAGGAAGA
SEQ ID NO. 153
AAGCCAGGTAAGGC
SEQ ID NO. 154
CCGCGGAAGGATAA
SEQ ID NO. 155
CATAATTCTATGCC
SEQ ID NO. 156
ATGAACTTGGCACA
SEQ ID NO. 157
CTTAAGCTTAAGAG
SEQ ID NO. 158
TCCTCGGAATGACA
SEQ ID NO. 159
AATCTTACCTAAGG
SEQ ID NO. 160
ACCACTGGTTGCCG
SEQ ID NO. 161
CTCCGTTCTGTACC
SEQ ID NO. 162
CCTAACTCGCCAGA
SEQ ID NO. 163
AATAGTGTTCAACC
SEQ ID NO. 164
CTACGAGTCATAGA
SEQ ID NO. 165
ACACACGACATCGC
SEQ ID NO. 166
AACTTACACGTTGG
SEQ ID NO. 167
AACACAACCTTCCG
SEQ ID NO. 168
ATACCTTAATCTCC
SEQ ID NO. 169
TAACCGCAATAGGA
SEQ ID NO. 170
CCGAACCATAGCCG
SEQ ID NO. 171
TTCATATAAGGCGC
SEQ ID NO. 172
AATACCAGCCGCGG
SEQ ID NO. 173
CTACGGACTAAGGC
SEQ ID NO. 174
TAATCGCCTACAGA
SEQ ID NO. 175
TTAGCCTAACTCGC
SEQ ID NO. 176
TAGTGTGGTAGACC
SEQ ID NO. 177
CAACGCCATAGACC
SEQ ID NO. 178
TAATCCATCAACGC
SEQ ID NO. 179
CCGGTGTGCGCTAA
SEQ ID NO. 180
TACCGGTATGGTCC
SEQ ID NO. 181
CACGTATAGATCGC
SEQ ID NO. 182
AACCTAAGCAATCC
SEQ ID NO. 183
CTAGTGTTCGATCC
SEQ ID NO. 184
CCTACACTTGAGCC
SEQ ID NO. 185
CAGCATCATACTGC
SEQ ID NO. 186
AATACGGAGACGGC
SEQ ID NO. 187
ATTGAACTCCATCC
SEQ ID NO. 188
AAGAGTCGGATAGG
SEQ ID NO. 189
AATGCAAGACAACC
SEQ ID NO. 190
TTCATTGGAAGAGA
SEQ ID NO. 191
TTCTAGGACGGCCG
SEQ ID NO. 192
CTCAACCACGTTCC
SEQ ID NO. 193
CTGGTCTGTCTTCC
SEQ ID NO. 194
TTCAAGTCAGTGCC
SEQ ID NO. 195
CACACGACAGAGCG
SEQ ID NO. 196
CCTAAGCCTGTTCG
SEQ ID NO. 197
AAGCTGGCGCCTAG
SEQ ID NO. 198
ACGCCTGTTGGAGG
SEQ ID NO. 199
TCTCCATTATACAG
SEQ ID NO. 200
TCCGCCGCACTCAA
SEQ ID NO. 201
TATCGATCATCTAC
SEQ ID NO. 202
TCTTGTACCGGACC
SEQ ID NO. 203
AACGGCTGAACGAC
SEQ ID NO. 204
TTCCGAAGCCTAAG
SEQ ID NO. 205
TTCTAGATAGGTAC
SEQ ID NO. 206
AATTCTCTGGCGCC
SEQ ID NO. 207
ATGACTGAGAGTGA
SEQ ID NO. 208
TAATTAGGTCCTCG
SEQ ID NO. 209
AACCGTCCAAGCGC
SEQ ID NO. 210
ACATTATTGGCCAA
SEQ ID NO. 211
TCCACAGAACCGGC
SEQ ID NO. 212
TAGTGATCTGTAGC
SEQ ID NO. 213
CATTCACGCTTGAA
SEQ ID NO. 214
CACACACGAGATGC
SEQ ID NO. 215
CCATGACCACCGGA
SEQ ID NO. 216
CAACAAGACCTAAG
SEQ ID NO. 217
AAGGAACCTCTCGG
SEQ ID NO. 218
CATAACCGGCACAA
SEQ ID NO. 219
CCAAGTGGTACAGC
SEQ ID NO. 220
TTCCTTACCGCGCG
SEQ ID NO. 221
ATACTAGTAGTCAG
SEQ ID NO. 222
TATATGCTCGCTCA
SEQ ID NO. 223
TCCGCGATCTCAAC
SEQ ID NO. 224
TATTACCGCTCAGG
SEQ ID NO. 225
ATCGAGCAGTGTAC
SEQ ID NO. 226
CATGCGTAGCGCAC
SEQ ID NO. 227
TCCAACAGTGTTGG
SEQ ID NO. 228
CACTTGGACCATCC
SEQ ID NO. 229
TTACCTGGCCATCC
SEQ ID NO. 230
CATCATGAGAGCGA
SEQ ID NO. 231
ATTCCGAATGTAAC
SEQ ID NO. 232
CCGGTATATCCGGA
SEQ ID NO. 233
ACTGACGTGGAACC
SEQ ID NO. 234
TTAAGCTGCCTTAA
SEQ ID NO. 235
CTCCGCAGTTGGAG
SEQ ID NO. 236
TCCTAATTCTAAGG
SEQ ID NO. 237
TACGATCGTGATCA
SEQ ID NO. 238
ATAACCAACTAGGA
SEQ ID NO. 239
ATCCTTATACGTGC
SEQ ID NO. 240
ACTTCCAGGCCTGA
SEQ ID NO. 241
TCTGCACATCACGC
SEQ ID NO. 242
ACAGACGCATCACG
SEQ ID NO. 243
TAAGTTGTGTGTCC
SEQ ID NO. 244
ACTCAACGGCAAGG
SEQ ID NO. 245
TAGCACATACTCGA
SEQ ID NO. 246
ATATCACAGTCGCA
SEQ ID NO. 247
ATATACACATGAGC
SEQ ID NO. 248
ACGTACGCGCATGC
SEQ ID NO. 249
CAAGCCAGTTCCGG
SEQ ID NO. 250
CCGAACAACCGTGA
SEQ ID NO. 251
CACACACATAGCGA
SEQ ID NO. 252
ACAGCGTACGCGCA
SEQ ID NO. 253
TCAACGCTCGGAGC
SEQ ID NO. 254
ACTACGATCATAGA
SEQ ID NO. 255
CAGGCGTAAGTGCC
SEQ ID NO. 256
ACATATCATGAGCA
SEQ ID NO. 257
AACTCCGTACAAGC
SEQ ID NO. 258
CTGTCGAGTAGCAG
SEQ ID NO. 259
TAGACGATAGTACA
SEQ ID NO. 260
CATGCTCACGCAGC
SEQ ID NO. 261
ATCACGAGCGACGC
SEQ ID NO. 262
CAGCCTGGCTTAGG
SEQ ID NO. 263
CCAGAGGTTGCCAG
SEQ ID NO. 264
TACTATGCACGACG
SEQ ID NO. 265
ACGCTGACGTGCGA
SEQ ID NO. 266
CAGCACATGTACAG
SEQ ID NO. 267
ACACGCATCATACG
SEQ ID NO. 268
CTGTACACGTCTAC
SEQ ID NO. 269
CACACAGTGAGTAC
SEQ ID NO. 270
TAGCACGCGATGAG
SEQ ID NO. 271
TCCAATAGGTCCAG
SEQ ID NO. 272
CTCCTGCGGTCCAA
SEQ ID NO. 273
ATGGCAACGGCTCA
SEQ ID NO. 274
CCGAGGCTTCCTAG
SEQ ID NO. 275
ACGACAGTAGTCGC
SEQ ID NO. 276
TTATAACTGGCAAC
SEQ ID NO. 277
ACTCTGAGTATCAG
SEQ ID NO. 278
TTATGTCTTAACGG
SEQ ID NO. 279
ATGCGACTCGAGAC
SEQ ID NO. 280
CCTCAATTCCGGCG
SEQ ID NO. 281
AATGGTTGGCAGAG
SEQ ID NO. 282
ATGCAAGCGATTCC
SEQ ID NO. 283
TATTGAGTCCGAGA
SEQ ID NO. 284
CCGCGTATTGCAAC
SEQ ID NO. 285
TCATTCTACTTGGC
SEQ ID NO. 286
ATCATCGCGCGACG
SEQ ID NO. 287
CAGAGACACTCGCA
SEQ ID NO. 288
AAGAAGAGTGGACC
SEQ ID NO. 289
ACACACGCTGCGAC
SEQ ID NO. 290
TCCGCAAGTCATAG
SEQ ID NO. 291
AATGTCCTCAGAAC
SEQ ID NO. 292
CCGAGTGTAAGTCC
SEQ ID NO. 293
ACACGTACTCATAC
SEQ ID NO. 294
TTCTCTCCGGTTCA
SEQ ID NO. 295
AATCCGTGTCTTAA
SEQ ID NO. 296
CCTTGCCAGAGTGG
SEQ ID NO. 297
CTTACTGACAAGCC
SEQ ID NO. 298
TCCTCCGATGCCAG
SEQ ID NO. 299
TTCTTCTCCAGAAG
SEQ ID NO. 300
CACCTGTTGGTTAA
SEQ ID NO. 301
CTCAGTCACACGAG
SEQ ID NO. 302
CCTTATCTACGACC
SEQ ID NO. 303
AACCGAACGCTTAA
SEQ ID NO. 304
ACAGGTAGGAAGGA
SEQ ID NO. 305
CAGCATAGTCTGAC
SEQ ID NO. 306
ATTCGGTCCGATGC
SEQ ID NO. 307
ACTAACCTGGCCGG
SEQ ID NO. 308
CATTCCTCTAGCCG
SEQ ID NO. 309
CTTAATCCATATCC
SEQ ID NO. 310
AACAAGAGGTGTGG
SEQ ID NO. 311
TATCATATGAGTCG
SEQ ID NO. 312
ACGAGTATGTATGC
SEQ ID NO. 313
TCGCGGATTAGGCA
SEQ ID NO. 314
CTACTGTGACACAC
SEQ ID NO. 315
CAACCGTGTGAACC
SEQ ID NO. 316
CAGCACTAGCTGCA
SEQ ID NO. 317
ACACGACATGCACG
SEQ ID NO. 318
ACTCATGTCAGACA
SEQ ID NO. 319
ACACTATACACGAG
SEQ ID NO. 320
ACTTGAGCAACCGG
SEQ ID NO. 321
TTAGACGCCGGTAA
SEQ ID NO. 322
CTGTGTAGGATTAA
SEQ ID NO. 323
CACGCATCAGTCAG
SEQ ID NO. 324
CAACCATTGGCAAG
SEQ ID NO. 325
TCATCTGCGCACAC
SEQ ID NO. 326
TAGGCCTCTGATGG
SEQ ID NO. 327
TAGCATGTCAGCAC
SEQ ID NO. 328
TCCTGGACGATGCC
SEQ ID NO. 329
ATATCACATGACAG
SEQ ID NO. 330
ACTACGTCGACACG
SEQ ID NO. 331
AATCCAACTCGAAG
SEQ ID NO. 332
AAGGATGCCAGTGG
SEQ ID NO. 333
ACCGCCTTGGCTAG
SEQ ID NO. 334
CTGTGATTCGGACC
SEQ ID NO. 335
CATGAGACGTCGCG
SEQ ID NO. 336
AATCCGCCTAATGG
SEQ ID NO. 337
AATGCCGTATTGAC
SEQ ID NO. 338
CTCCGAAGAACCGG
SEQ ID NO. 339
ACTGATTAGAATCG
SEQ ID NO. 340
ACTCGCCACCGAAG
SEQ ID NO. 341
CCGGTATTGTTACA
SEQ ID NO. 342
TTGGTGAGAACCGC
SEQ ID NO. 343
CAGATCTCAGACAC
SEQ ID NO. 344
CCTTAACACGGAGA
SEQ ID NO. 345
ATGTCTAGTATAGC
SEQ ID NO. 346
CCAGAGGTAATGGA
SEQ ID NO. 347
CTGTCACACAGTCA
SEQ ID NO. 348
TCACTGAGTGCTGA
SEQ ID NO. 349
TCCTATGGTAGGAG
SEQ ID NO. 350
AAGGAACAATACAC
SEQ ID NO. 351
TAAGCCGGAATTAG
SEQ ID NO. 352
ATAATGCTCCAGAA
SEQ ID NO. 353
ATTCTTGCGAAGCG
SEQ ID NO. 354
ACATGCTATACTAC
SEQ ID NO. 355
AAGCCAACAGTTGG
SEQ ID NO. 356
TATGAGCACACGAC
SEQ ID NO. 357
CACTAGATATCACA
SEQ ID NO. 358
TCAATTAAGGCACG
SEQ ID NO. 359
TCCTCCTCTTGTGA
SEQ ID NO. 360
TACTACTATGTGAC
SEQ ID NO. 361
TATCTTCCACCTGA
SEQ ID NO. 362
CACTTACCTTACAA
SEQ ID NO. 363
TCTAGCATCGACGA
SEQ ID NO. 364
ACACGAGCACGAGC
SEQ ID NO. 365
ACAAGGCAACAGCC
SEQ ID NO. 366
CACTACGCGCTCAC
SEQ ID NO. 367
TAACCGTCTCTGGC
SEQ ID NO. 368
CCAACTCGCCAGGA
SEQ ID NO. 369
CTTCGGTACACTAA
SEQ ID NO. 370
CCACACCTTCAGGC
SEQ ID NO. 371
CCACTTCTTGTTGG
SEQ ID NO. 372
CTAGTCTTGGTAGG
SEQ ID NO. 373
TCAGAATCCTCGCC
SEQ ID NO. 374
CTAGGAGCTTGAAC
SEQ ID NO. 375
CCGGCGCAATTCAG
SEQ ID NO. 376
ACCGGTCGTTCTGC
SEQ ID NO. 377
CATTAAGGTATCGC
SEQ ID NO. 378
CTTGGAGTGGCACA
SEQ ID NO. 379
AACCTTCTAGAGAA
SEQ ID NO. 380
CATGGAATGTGTAA
SEQ ID NO. 381
CTGTTCGGAGGTCA
SEQ ID NO. 382
ATCGTGTGTGGCCA
SEQ ID NO. 383
TTGTCGGTTGAGGC
SEQ ID NO. 384
CTGGCGTTGAGTCG
SEQ ID NO. 385
AATGACCACGACCG
SEQ ID NO. 386
ATGTCTACAGACGA
SEQ ID NO. 387
TCTCCGAGGTAGGC
SEQ ID NO. 388
ACGACGACTGACAG
SEQ ID NO. 389
ACTATGACTGCGCA
SEQ ID NO. 390
CCGCCTTCTAACAC
SEQ ID NO. 391
CACACGCAGTATCA
SEQ ID NO. 392
CCAGCTTAGGAAGA
SEQ ID NO. 393
ACACGATCTGACGA
SEQ ID NO. 394
AAGGCAAGTTGTGA
SEQ ID NO. 395
AACTCTTCGGTAGG
SEQ ID NO. 396
CCGTCCTTCTTCAC
SEQ ID NO. 397
CTTGGTTGGTTCCG
SEQ ID NO. 398
TACCTTGGAGTTGG
SEQ ID NO. 399
ACCGGCGGTATAGG
SEQ ID NO. 400
CACGACGACTCAGA
SEQ ID NO. 401
TCTTGTGAGGTTCG
SEQ ID NO. 402
AACGCTTCCGGTCC
SEQ ID NO. 403
CTACTGCATAGTAG
SEQ ID NO. 404
ACACAGCACGATAG
SEQ ID NO. 405
CCTACAGGTCGAGG
SEQ ID NO. 406
ATGCACGATCGAGC
SEQ ID NO. 407
CTTGATACCGGCGC
SEQ ID NO. 408
CCTCGGTTGAAGCC
SEQ ID NO. 409
CAACACCGGCTTGG
SEQ ID NO. 410
CTCTGGACCGATCA
SEQ ID NO. 411
CCGTGAACCTTGCG
SEQ ID NO. 412
TCATGCTCGAGACA
SEQ ID NO. 413
ACCGGAACCTATGG
SEQ ID NO. 414
TTCCAAGGTTGACA
SEQ ID NO. 415
ACGAGAGTGCTGAG
SEQ ID NO. 416
CTCAACCGGACGGA
SEQ ID NO. 417
AATCAAGTGTGAAC
SEQ ID NO. 418
CATACGGCCTCTAA
SEQ ID NO. 419
ACTAGAGCGTGTGA
SEQ ID NO. 420
TCAGAGTAGTGCAG
SEQ ID NO. 421
CAACAACCTTGAGG
SEQ ID NO. 422
ACAATTAGCCTTGG
SEQ ID NO. 423
TCACTATGCGATAC
SEQ ID NO. 424
TACTCTCATCTAGC
SEQ ID NO. 425
TCTCTCTCAATTGG
SEQ ID NO. 426
CCAGTGAGGTATAC
SEQ ID NO. 427
CTCCTACTTAATGC
SEQ ID NO. 428
ATAGGCCTTCGAGG
SEQ ID NO. 429
CATTGGTGGTGTCC
SEQ ID NO. 430
CATACTATGTAGAC
SEQ ID NO. 431
ATGGATCGTTAGGA
SEQ ID NO. 432
CCAACCGAACGGCA
SEQ ID NO. 433
ACGTACATCTATCG
SEQ ID NO. 434
TACGAGTGTCTCAG
SEQ ID NO. 435
ATAACAACGAAGCC
SEQ ID NO. 436
TACGATAGTACAGC
SEQ ID NO. 437
ATTGCGGTTAATCA
SEQ ID NO. 438
CAGTTGCGAAGTGG
SEQ ID NO. 439
TTAACACCTCAAGG
SEQ ID NO. 440
CACGACATGCGAGC
SEQ ID NO. 441
TCCAGAACTGAGGC
SEQ ID NO. 442
TATTCCTAGTTCGA
SEQ ID NO. 443
AACACCTTCTCCGC
SEQ ID NO. 444
TAACCTGTTCAAGA
SEQ ID NO. 445
AAGCTATGTGCAAC
SEQ ID NO. 446
CTCTTAATCCTTGA
SEQ ID NO. 447
ATCAGCAGACTAGC
SEQ ID NO. 448
CAAGGAAGCATTGG
SEQ ID NO. 449
ACCTCGAAGTTCAA
SEQ ID NO. 450
ATAGCACTCACACG
SEQ ID NO. 451
ATCTAGCTGCACGA
SEQ ID NO. 452
TCTGCTGCAGAGCG
SEQ ID NO. 453
TAACAGTCCGTTCA
SEQ ID NO. 454
CATCTATCTGCGCG
SEQ ID NO. 455
TTGACCAAGATTAC
SEQ ID NO. 456
TCTCACATCACGAG
SEQ ID NO. 457
ACACATCTCTACGA
SEQ ID NO. 458
AACAGAATAGGCGG
SEQ ID NO. 459
AACACCTAAGGAAG
SEQ ID NO. 460
AACACCTGGTTGAA
SEQ ID NO. 461
AAGACGAATAGGAA
SEQ ID NO. 462
CAAGTTCAGAATGG
SEQ ID NO. 463
CCAAGCTTAGTGCG
SEQ ID NO. 464
AACGCTCAATGTGG
SEQ ID NO. 465
TCATCTAGGTGGAA
SEQ ID NO. 466
CCTGTGATTACACC
SEQ ID NO. 467
TCAGAACAGGTTAA
SEQ ID NO. 468
ATATTGTAAGGTGG
SEQ ID NO. 469
TTAAGTGACCGCGC
SEQ ID NO. 470
AATCCTGAATCCAA
SEQ ID NO. 471
CTATAGCGCACTAC
SEQ ID NO. 472
TCTAGACACTATCG
SEQ ID NO. 473
TCAATGGCAGGCCG
SEQ ID NO. 474
AAGTGGTATTGACA
SEQ ID NO. 475
TTGGACATTCCGGC
SEQ ID NO. 476
CAGCATCGAGAGCG
SEQ ID NO. 477
CAAGACCTTGGCCA
SEQ ID NO. 478
CCGGTTCCTAGTGA
SEQ ID NO. 479
ACAACTTAGCCTAA
SEQ ID NO. 480
TCGTGTCGCATGCA
SEQ ID NO. 481
CACACAGACGCTCG,
and a combination of one or more thereof.

8. The one or more oligonucleotide molecules of claim 1, consisting of at least 10 oligonucleotide molecules, at least 20 oligonucleotide molecules, at least 30 oligonucleotide molecules, at least 40 oligonucleotide molecules, at least 50 oligonucleotide molecules, at least 60 oligonucleotide molecules, at least 70 oligonucleotide molecules, at least 80 oligonucleotide molecules, at least 90 oligonucleotide molecules, at least 100 oligonucleotide molecules, at least 150 oligonucleotide molecules, at least 200 oligonucleotide molecules, at least 250 oligonucleotide molecules, at least 300 oligonucleotide molecules, or 384 oligonucleotides molecules.

9. The one or more oligonucleotide molecules of claim 2, wherein the UMI consists of a sequence (N)n(V)m, wherein N is any nucleotide selected from A, T, C and G; V is any nucleotide selected from A, C and G; n is an integer selected from 1 to 20, and m is an integer selected from 1 to 20.

10. The one or more oligonucleotide molecules of claim 1, wherein the mRNA capture sequence is a poly-T sequence followed by at least one V and one N, wherein N is any nucleotide selected from A, T, C and G; and V is any nucleotide selected from A, C and G.

11. A set of oligonucleotide molecules, each molecule comprising, from 5′ to 3′,
a) a sequencing adaptor,
b) a barcode sequence independently selected from the group consisting of SEQ ID: 1 to 96 or a barcode sequence independently selected from the group consisting of SEQ ID: 98 to 481, and
c) an mRNA capture sequence.

12. The set of oligonucleotide molecules of claim 11, further comprising d) a UMI.

13. A barcode oligonucleotide sequence selected from the group consisting of a sequence as set forth in SEQ ID NO. 1 to SEQ ID NO. 96, or a combination of one or more thereof.

14. A barcode oligonucleotide sequence selected from the group consisting of a sequence as set forth in SEQ ID NO. 98 to SEQ ID NO. 481, or a combination of one or more thereof.

15. (canceled)

16. A method for providing a cDNA library, the method comprising the steps of:
a) Providing a plurality of RNA samples obtained from a biological sample;
b) Contacting separately each RNA sample with one or more oligonucleotide molecules of claim 1 under annealing conditions;
c) Incubating separately each sample under reverse transcription reaction conditions;
d) Pooling together all first strand cDNA or cDNA:RNA sample;
e) Proceeding to second strand synthesis under synthesis conditions; and
f) Proceeding with tagmentation and amplification under suitable conditions to obtain a sequencing compatible cDNA library; or
g) Proceeding with enzymatic and physical force fragmentation followed by DNA end repair, A-tailing, DNA adapter ligation and amplification under suitable conditions to obtain a sequencing compatible cDNA library.

17. The method for providing a cDNA library of claim 16, wherein the second strand synthesis is generated by a method selected from the group consisting of PCR amplification and nick translation, or a combination thereof.

18. A method for sequencing RNA, the method comprising the steps of:
a) Providing a cDNA library obtained by the method of claim 16; and
b) Proceeding to the sequencing under suitable conditions.

19. A kit comprising:
a) one or more oligonucleotide molecules of claim 1,
b) a support for sample preparation, and
c) reagents for sequencing.

20. The kit of claim 19 further comprising instructions for single-cell RNA profiling.


SEQUENCE LISTING (485 sequences)

SEQ ID NO: 1 - DNA, 14 bp, Artificial Sequence
  Designed nucleotides based on the discovery of an optimal set barcoded oligonucleotides for multiplexed RNA sequencing.
tacgttattccgaa

SEQ ID NO: 2 - DNA, 14 bp, Artificial Sequence
  Designed nucleotides based on the discovery of an optimal set barcoded oligonucleotides for multiplexed RNA sequencing.
aacaggataactcc

SEQ ID NO: 3 - DNA, 14 bp, Artificial Sequence
  Designed nucleotides based on the discovery of an optimal set barcoded oligonucleotides for multiplexed RNA sequencing.
actcaggcacctcc

SEQ ID NO: 4 - DNA, 14 bp, Artificial Sequence
  Designed nucleotides based on the discovery of an optimal set barcoded oligonucleotides for multiplexed RNA sequencing.
acgagcagatgcag

SEQ ID NO: 5 - DNA, 14 bp, Artificial Sequence
  Designed nucleotides based on the discovery of an optimal set barcoded oligonucleotides for multiplexed RNA sequencing.
ttcaatctccttag

SEQ ID NO: 6 - DNA, 14 bp, Artificial Sequence
  Designed nucleotides based on the discovery of an optimal set barcoded oligonucleotides for multiplexed RNA sequencing.
ctcggttcgaatgc

SEQ ID NO: 7 - DNA, 14 bp, Artificial Sequence
  Designed nucleotides based on the discovery of an optimal set barcoded oligonucleotides for multiplexed RNA sequencing.
tacactatagctag

SEQ ID NO: 8 - DNA, 14 bp, Artificial Sequence
  Designed nucleotides based on the discovery of an optimal set barcoded oligonucleotides for multiplexed RNA sequencing.
caagtataaggaac

SEQ ID NO: 9 - DNA, 14 bp, Artificial Sequence
  Designed nucleotides based on the discovery of an optimal set barcoded oligonucleotides for multiplexed RNA sequencing.
ctgatatgcagcga

SEQ ID NO: 10 - DNA, 14 bp, Artificial Sequence
  Designed nucleotides based on the discovery of an optimal set barcoded oligonucleotides for multiplexed RNA sequencing.
acaataggtggtcc

SEQ ID NO: 11 - DNA, 14 bp, Artificial Sequence
  Designed nucleotides based on the discovery of an optimal set barcoded oligonucleotides for multiplexed RNA sequencing.
aaccatcgcatcca

SEQ ID NO: 12 - DNA, 14 bp, Artificial Sequence
  Designed nucleotides based on the discovery of an optimal set barcoded oligonucleotides for multiplexed RNA sequencing.
cagtctagtgcgca

SEQ ID NO: 13 - DNA, 14 bp, Artificial Sequence
  Designed nucleotides based on the discovery of an optimal set barcoded oligonucleotides for multiplexed RNA sequencing.
ccgcaagaggttgg

SEQ ID NO: 14 - DNA, 14 bp, Artificial Sequence
  Designed nucleotides based on the discovery of an optimal set barcoded oligonucleotides for multiplexed RNA sequencing.
taacacctccaacg

SEQ ID NO: 15 - DNA, 14 bp, Artificial Sequence
  Designed nucleotides based on the discovery of an optimal set barcoded oligonucleotides for multiplexed RNA sequencing.
cagacatgatagag

SEQ ID NO: 16 - DNA, 14 bp, Artificial Sequence
  Designed nucleotides based on the discovery of an optimal set barcoded oligonucleotides for multiplexed RNA sequencing.
aacaaccgaagtaa

SEQ ID NO: 17 - DNA, 14 bp, Artificial Sequence
  Designed nucleotides based on the discovery of an optimal set barcoded oligonucleotides for multiplexed RNA sequencing.
aactagtatccgga

SEQ ID NO: 18 - DNA, 14 bp, Artificial Sequence
  Designed nucleotides based on the discovery of an optimal set barcoded oligonucleotides for multiplexed RNA sequencing.
acgcagcgagcaga

SEQ ID NO: 19 - DNA, 14 bp, Artificial Sequence
  Designed nucleotides based on the discovery of an optimal set barcoded oligonucleotides for multiplexed RNA sequencing.
tccaaggcattaag

SEQ ID NO: 20 - DNA, 14 bp, Artificial Sequence
  Designed nucleotides based on the discovery of an optimal set barcoded oligonucleotides for multiplexed RNA sequencing.
tatgactgcatgcg

SEQ ID NO: 21 - DNA, 14 bp, Artificial Sequence
  Designed nucleotides based on the discovery of an optimal set barcoded oligonucleotides for multiplexed RNA sequencing.
accggataacggaa

SEQ ID NO: 22 - DNA, 14 bp, Artificial Sequence
  Designed nucleotides based on the discovery of an optimal set barcoded oligonucleotides for multiplexed RNA sequencing.
ataatattgcctcc

SEQ ID NO: 23 - DNA, 14 bp, Artificial Sequence
  Designed nucleotides based on the discovery of an optimal set barcoded oligonucleotides for multiplexed RNA sequencing.
tcttgaacgactaa

SEQ ID NO: 24 - DNA, 14 bp, Artificial Sequence
  Designed nucleotides based on the discovery of an optimal set barcoded oligonucleotides for multiplexed RNA sequencing.
ttaacttacggtca

SEQ ID NO: 25 - DNA, 14 bp, Artificial Sequence
  Designed nucleotides based on the discovery of an optimal set barcoded oligonucleotides for multiplexed RNA sequencing.
catgctagcgatcg

SEQ ID NO: 26 - DNA, 14 bp, Artificial Sequence
  Designed nucleotides based on the discovery of an optimal set barcoded oligonucleotides for multiplexed RNA sequencing.
taagcaacagccga

SEQ ID NO: 27 - DNA, 14 bp, Artificial Sequence
  Designed nucleotides based on the discovery of an optimal set barcoded oligonucleotides for multiplexed RNA sequencing.
atgtaggtaatcgg

SEQ ID NO: 28 - DNA, 14 bp, Artificial Sequence
  Designed nucleotides based on the discovery of an optimal set barcoded oligonucleotides for multiplexed RNA sequencing.
cagcagcgctgaca

SEQ ID NO: 29 - DNA, 14 bp, Artificial Sequence
  Designed nucleotides based on the discovery of an optimal set barcoded oligonucleotides for multiplexed RNA sequencing.
tatgtacgtagaga

SEQ ID NO: 30 - DNA, 14 bp, Artificial Sequence
  Designed nucleotides based on the discovery of an optimal set barcoded oligonucleotides for multiplexed RNA sequencing.
cagatcgctctgcg

SEQ ID NO: 31 - DNA, 14 bp, Artificial Sequence
  Designed nucleotides based on the discovery of an optimal set barcoded oligonucleotides for multiplexed RNA sequencing.
taacctaacttcac

SEQ ID NO: 32 - DNA, 14 bp, Artificial Sequence
  Designed nucleotides based on the discovery of an optimal set barcoded oligonucleotides for multiplexed RNA sequencing.
ctcctaacacagcc

SEQ ID NO: 33 - DNA, 14 bp, Artificial Sequence
  Designed nucleotides based on the discovery of an optimal set barcoded oligonucleotides for multiplexed RNA sequencing.
ataccgttggagga

SEQ ID NO: 34 - DNA, 14 bp, Artificial Sequence
  Designed nucleotides based on the discovery of an optimal set barcoded oligonucleotides for multiplexed RNA sequencing.
cttatatcgtcggc

SEQ ID NO: 35 - DNA, 14 bp, Artificial Sequence
  Designed nucleotides based on the discovery of an optimal set barcoded oligonucleotides for multiplexed RNA sequencing.
aagtactgatccaa

SEQ ID NO: 36 - DNA, 14 bp, Artificial Sequence
  Designed nucleotides based on the discovery of an optimal set barcoded oligonucleotides for multiplexed RNA sequencing.
tactgactatcgag

SEQ ID NO: 37 - DNA, 14 bp, Artificial Sequence
  Designed nucleotides based on the discovery of an optimal set barcoded oligonucleotides for multiplexed RNA sequencing.
attgcgtccattag

SEQ ID NO: 38 - DNA, 14 bp, Artificial Sequence
  Designed nucleotides based on the discovery of an optimal set barcoded oligonucleotides for multiplexed RNA sequencing.
ttgctaccattgaa

SEQ ID NO: 39 - DNA, 14 bp, Artificial Sequence
  Designed nucleotides based on the discovery of an optimal set barcoded oligonucleotides for multiplexed RNA sequencing.
aagcgcaccggaac

SEQ ID NO: 40 - DNA, 14 bp, Artificial Sequence
  Designed nucleotides based on the discovery of an optimal set barcoded oligonucleotides for multiplexed RNA sequencing.
accaacttagacaa

SEQ ID NO: 41 - DNA, 14 bp, Artificial Sequence
  Designed nucleotides based on the discovery of an optimal set barcoded oligonucleotides for multiplexed RNA sequencing.
tcagcgtgtcgtca

SEQ ID NO: 42 - DNA, 14 bp, Artificial Sequence
  Designed nucleotides based on the discovery of an optimal set barcoded oligonucleotides for multiplexed RNA sequencing.
attccgctctgagg

SEQ ID NO: 43 - DNA, 14 bp, Artificial Sequence
  Designed nucleotides based on the discovery of an optimal set barcoded oligonucleotides for multiplexed RNA sequencing.
accgatgtgccgga

SEQ ID NO: 44 - DNA, 14 bp, Artificial Sequence
  Designed nucleotides based on the discovery of an optimal set barcoded oligonucleotides for multiplexed RNA sequencing.
ctaaggaaggaccg

SEQ ID NO: 45 - DNA, 14 bp, Artificial Sequence
  Designed nucleotides based on the discovery of an optimal set barcoded oligonucleotides for multiplexed RNA sequencing.
ttctgaggcgatgg

SEQ ID NO: 46 - DNA, 14 bp, Artificial Sequence
  Designed nucleotides based on the discovery of an optimal set barcoded oligonucleotides for multiplexed RNA sequencing.
acatcttcttgaac

SEQ ID NO: 47 - DNA, 14 bp, Artificial Sequence
  Designed nucleotides based on the discovery of an optimal set barcoded oligonucleotides for multiplexed RNA sequencing.
acagacgatagtca

SEQ ID NO: 48 - DNA, 14 bp, Artificial Sequence
  Designed nucleotides based on the discovery of an optimal set barcoded oligonucleotides for multiplexed RNA sequencing.
actatctgcagtcg

SEQ ID NO: 49 - DNA, 14 bp, Artificial Sequence
  Designed nucleotides based on the discovery of an optimal set barcoded oligonucleotides for multiplexed RNA sequencing.
catgtgtactagca

SEQ ID NO: 50 - DNA, 14 bp, Artificial Sequence
  Designed nucleotides based on the discovery of an optimal set barcoded oligonucleotides for multiplexed RNA sequencing.
ctctggcttgcagg

SEQ ID NO: 51 - DNA, 14 bp, Artificial Sequence
  Designed nucleotides based on the discovery of an optimal set barcoded oligonucleotides for multiplexed RNA sequencing.
acggccttcaacca

SEQ ID NO: 52 - DNA, 14 bp, Artificial Sequence
  Designed nucleotides based on the discovery of an optimal set barcoded oligonucleotides for multiplexed RNA sequencing.
ccatattgaagcac

SEQ ID NO: 53 - DNA, 14 bp, Artificial Sequence
  Designed nucleotides based on the discovery of an optimal set barcoded oligonucleotides for multiplexed RNA sequencing.
tcgattggtaatgg

SEQ ID NO: 54 - DNA, 14 bp, Artificial Sequence
  Designed nucleotides based on the discovery of an optimal set barcoded oligonucleotides for multiplexed RNA sequencing.
ttcatggtagagag

SEQ ID NO: 55 - DNA, 14 bp, Artificial Sequence
  Designed nucleotides based on the discovery of an optimal set barcoded oligonucleotides for multiplexed RNA sequencing.
acgctcctaacggc

SEQ ID NO: 56 - DNA, 14 bp, Artificial Sequence
  Designed nucleotides based on the discovery of an optimal set barcoded oligonucleotides for multiplexed RNA sequencing.
ctcaattagagacc

SEQ ID NO: 57 - DNA, 14 bp, Artificial Sequence
  Designed nucleotides based on the discovery of an optimal set barcoded oligonucleotides for multiplexed RNA sequencing.
tcgatcttcggcag

SEQ ID NO: 58 - DNA, 14 bp, Artificial Sequence
  Designed nucleotides based on the discovery of an optimal set barcoded oligonucleotides for multiplexed RNA sequencing.
cacatcgacagcag

SEQ ID NO: 59 - DNA, 14 bp, Artificial Sequence
  Designed nucleotides based on the discovery of an optimal set barcoded oligonucleotides for multiplexed RNA sequencing.
aacctgctatccgg

SEQ ID NO: 60 - DNA, 14 bp, Artificial Sequence
  Designed nucleotides based on the discovery of an optimal set barcoded oligonucleotides for multiplexed RNA sequencing.
atccggcgatgacc

SEQ ID NO: 61 - DNA, 14 bp, Artificial Sequence
  Designed nucleotides based on the discovery of an optimal set barcoded oligonucleotides for multiplexed RNA sequencing.
tccatgtgtccagg

SEQ ID NO: 62 - DNA, 14 bp, Artificial Sequence
  Designed nucleotides based on the discovery of an optimal set barcoded oligonucleotides for multiplexed RNA sequencing.
atcgatgtcatagc

SEQ ID NO: 63 - DNA, 14 bp, Artificial Sequence
  Designed nucleotides based on the discovery of an optimal set barcoded oligonucleotides for multiplexed RNA sequencing.
ctccgcatgcttcc

SEQ ID NO: 64 - DNA, 14 bp, Artificial Sequence
  Designed nucleotides based on the discovery of an optimal set barcoded oligonucleotides for multiplexed RNA sequencing.
cacgtagctagacg

SEQ ID NO: 65 - DNA, 14 bp, Artificial Sequence
  Designed nucleotides based on the discovery of an optimal set barcoded oligonucleotides for multiplexed RNA sequencing.
aacatggcacggaa

SEQ ID NO: 66 - DNA, 14 bp, Artificial Sequence
  Designed nucleotides based on the discovery of an optimal set barcoded oligonucleotides for multiplexed RNA sequencing.
atgctatctagaca

SEQ ID NO: 67 - DNA, 14 bp, Artificial Sequence
  Designed nucleotides based on the discovery of an optimal set barcoded oligonucleotides for multiplexed RNA sequencing.
accaatccgtataa

SEQ ID NO: 68 - DNA, 14 bp, Artificial Sequence
  Designed nucleotides based on the discovery of an optimal set barcoded oligonucleotides for multiplexed RNA sequencing.
ctcgtgcctcagga

SEQ ID NO: 69 - DNA, 14 bp, Artificial Sequence
  Designed nucleotides based on the discovery of an optimal set barcoded oligonucleotides for multiplexed RNA sequencing.
atgcaattacaccg

SEQ ID NO: 70 - DNA, 14 bp, Artificial Sequence
  Designed nucleotides based on the discovery of an optimal set barcoded oligonucleotides for multiplexed RNA sequencing.
cctccatccatgga

SEQ ID NO: 71 - DNA, 14 bp, Artificial Sequence
  Designed nucleotides based on the discovery of an optimal set barcoded oligonucleotides for multiplexed RNA sequencing.
acggctcttaggcc

SEQ ID NO: 72 - DNA, 14 bp, Artificial Sequence
  Designed nucleotides based on the discovery of an optimal set barcoded oligonucleotides for multiplexed RNA sequencing.
tccaactaccgaac

SEQ ID NO: 73 - DNA, 14 bp, Artificial Sequence
  Designed nucleotides based on the discovery of an optimal set barcoded oligonucleotides for multiplexed RNA sequencing.
tatgagagctatga

SEQ ID NO: 74 - DNA, 14 bp, Artificial Sequence
  Designed nucleotides based on the discovery of an optimal set barcoded oligonucleotides for multiplexed RNA sequencing.
caaggcgtggttac

SEQ ID NO: 75 - DNA, 14 bp, Artificial Sequence
  Designed nucleotides based on the discovery of an optimal set barcoded oligonucleotides for multiplexed RNA sequencing.
attatcggattcgg

SEQ ID NO: 76 - DNA, 14 bp, Artificial Sequence
  Designed nucleotides based on the discovery of an optimal set barcoded oligonucleotides for multiplexed RNA sequencing.
atcctaagtggagg

SEQ ID NO: 77 - DNA, 14 bp, Artificial Sequence
  Designed nucleotides based on the discovery of an optimal set barcoded oligonucleotides for multiplexed RNA sequencing.
atacgatgctgcgc

SEQ ID NO: 78 - DNA, 14 bp, Artificial Sequence
  Designed nucleotides based on the discovery of an optimal set barcoded oligonucleotides for multiplexed RNA sequencing.
aagccttaggccgc

SEQ ID NO: 79 - DNA, 14 bp, Artificial Sequence
  Designed nucleotides based on the discovery of an optimal set barcoded oligonucleotides for multiplexed RNA sequencing.
aatgtgtggatacc

SEQ ID NO: 80 - DNA, 14 bp, Artificial Sequence
  Designed nucleotides based on the discovery of an optimal set barcoded oligonucleotides for multiplexed RNA sequencing.
atgctgcaacggca

SEQ ID NO: 81 - DNA, 14 bp, Artificial Sequence
  Designed nucleotides based on the discovery of an optimal set barcoded oligonucleotides for multiplexed RNA sequencing.
cttgataagccagg

SEQ ID NO: 82 - DNA, 14 bp, Artificial Sequence
  Designed nucleotides based on the discovery of an optimal set barcoded oligonucleotides for multiplexed RNA sequencing.
catggaactccggc

SEQ ID NO: 83 - DNA, 14 bp, Artificial Sequence
  Designed nucleotides based on the discovery of an optimal set barcoded oligonucleotides for multiplexed RNA sequencing.
taaggacctcataa

SEQ ID NO: 84 - DNA, 14 bp, Artificial Sequence
  Designed nucleotides based on the discovery of an optimal set barcoded oligonucleotides for multiplexed RNA sequencing.
tctgtctatgcagc

SEQ ID NO: 85 - DNA, 14 bp, Artificial Sequence
  Designed nucleotides based on the discovery of an optimal set barcoded oligonucleotides for multiplexed RNA sequencing.
caaccaacctatag

SEQ ID NO: 86 - DNA, 14 bp, Artificial Sequence
  Designed nucleotides based on the discovery of an optimal set barcoded oligonucleotides for multiplexed RNA sequencing.
ccggtagaatggcc

SEQ ID NO: 87 - DNA, 14 bp, Artificial Sequence
  Designed nucleotides based on the discovery of an optimal set barcoded oligonucleotides for multiplexed RNA sequencing.
tccgaagttgtaga

SEQ ID NO: 88 - DNA, 14 bp, Artificial Sequence
  Designed nucleotides based on the discovery of an optimal set barcoded oligonucleotides for multiplexed RNA sequencing.
tcgatgctatatga

SEQ ID NO: 89 - DNA, 14 bp, Artificial Sequence
  Designed nucleotides based on the discovery of an optimal set barcoded oligonucleotides for multiplexed RNA sequencing.
acaaggttccgccg

SEQ ID NO: 90 - DNA, 14 bp, Artificial Sequence
  Designed nucleotides based on the discovery of an optimal set barcoded oligonucleotides for multiplexed RNA sequencing.
ccacgttccggtag

SEQ ID NO: 91 - DNA, 14 bp, Artificial Sequence
  Designed nucleotides based on the discovery of an optimal set barcoded oligonucleotides for multiplexed RNA sequencing.
acttaaggtccaac

SEQ ID NO: 92 - DNA, 14 bp, Artificial Sequence
  Designed nucleotides based on the discovery of an optimal set barcoded oligonucleotides for multiplexed RNA sequencing.
tccagaaggcaaca

SEQ ID NO: 93 - DNA, 14 bp, Artificial Sequence
  Designed nucleotides based on the discovery of an optimal set barcoded oligonucleotides for multiplexed RNA sequencing.
ctgctcatgagaga

SEQ ID NO: 94 - DNA, 14 bp, Artificial Sequence
  Designed nucleotides based on the discovery of an optimal set barcoded oligonucleotides for multiplexed RNA sequencing.
atgaatgttgccac

SEQ ID NO: 95 - DNA, 14 bp, Artificial Sequence
  Designed nucleotides based on the discovery of an optimal set barcoded oligonucleotides for multiplexed RNA sequencing.
ctctcaagtcctcc

SEQ ID NO: 96 - DNA, 14 bp, Artificial Sequence
  Designed nucleotides based on the discovery of an optimal set barcoded oligonucleotides for multiplexed RNA sequencing.
attccagttcttgc

SEQ ID NO: 97 - DNA, 22 bp, Artificial Sequence
  Designed nucleotides based on the discovery of an optimal set barcoded oligonucleotides for multiplexed RNA sequencing.
ctacacgacgctcttccgatct

SEQ ID NO: 98 - DNA, 14 bp, Artificial Sequence
  Designed nucleotides based on the discovery of an optimal set barcoded oligonucleotides for multiplexed RNA sequencing.
tcgtacatagcgca

SEQ ID NO: 99 - DNA, 14 bp, Artificial Sequence
  Designed nucleotides based on the discovery of an optimal set barcoded oligonucleotides for multiplexed RNA sequencing.
caaggaaccgaacc

SEQ ID NO: 100 - DNA, 14 bp, Artificial Sequence
  Designed nucleotides based on the discovery of an optimal set barcoded oligonucleotides for multiplexed RNA sequencing.
tcagtcacgcgtgc

SEQ ID NO: 101 - DNA, 14 bp, Artificial Sequence
  Designed nucleotides based on the discovery of an optimal set barcoded oligonucleotides for multiplexed RNA sequencing.
cacacgatactcac

SEQ ID NO: 102 - DNA, 14 bp, Artificial Sequence
  Designed nucleotides based on the discovery of an optimal set barcoded oligonucleotides for multiplexed RNA sequencing.
aaggagaagaatgc

SEQ ID NO: 103 - DNA, 14 bp, Artificial Sequence
  Designed nucleotides based on the discovery of an optimal set barcoded oligonucleotides for multiplexed RNA sequencing.
cagtggcctaagca

SEQ ID NO: 104 - DNA, 14 bp, Artificial Sequence
  Designed nucleotides based on the discovery of an optimal set barcoded oligonucleotides for multiplexed RNA sequencing.
accgtcttccttgc

SEQ ID NO: 105 - DNA, 14 bp, Artificial Sequence
  Designed nucleotides based on the discovery of an optimal set barcoded oligonucleotides for multiplexed RNA sequencing.
cctcattcgttgag

SEQ ID NO: 106 - DNA, 14 bp, Artificial Sequence
  Designed nucleotides based on the discovery of an optimal set barcoded oligonucleotides for multiplexed RNA sequencing.
acgatatgctctgc

SEQ ID NO: 107 - DNA, 14 bp, Artificial Sequence
  Designed nucleotides based on the discovery of an optimal set barcoded oligonucleotides for multiplexed RNA sequencing.
ccaccgttaagaca

SEQ ID NO: 108 - DNA, 14 bp, Artificial Sequence
  Designed nucleotides based on the discovery of an optimal set barcoded oligonucleotides for multiplexed RNA sequencing.
actactcgatctag

SEQ ID NO: 109 - DNA, 14 bp, Artificial Sequence
  Designed nucleotides based on the discovery of an optimal set barcoded oligonucleotides for multiplexed RNA sequencing.
aacacttggccacc

SEQ ID NO: 110 - DNA, 14 bp, Artificial Sequence
  Designed nucleotides based on the discovery of an optimal set barcoded oligonucleotides for multiplexed RNA sequencing.
atcacatctatcgc

SEQ ID NO: 111 - DNA, 14 bp, Artificial Sequence
  Designed nucleotides based on the discovery of an optimal set barcoded oligonucleotides for multiplexed RNA sequencing.
ctccaatattcaac

SEQ ID NO: 112 - DNA, 14 bp, Artificial Sequence
  Designed nucleotides based on the discovery of an optimal set barcoded oligonucleotides for multiplexed RNA sequencing.
ccaatagacttccg

SEQ ID NO: 113 - DNA, 14 bp, Artificial Sequence
  Designed nucleotides based on the discovery of an optimal set barcoded oligonucleotides for multiplexed RNA sequencing.
accgtaacgtggac

SEQ ID NO: 114 - DNA, 14 bp, Artificial Sequence
  Designed nucleotides based on the discovery of an optimal set barcoded oligonucleotides for multiplexed RNA sequencing.
ataaggaccggagg

SEQ ID NO: 115 - DNA, 14 bp, Artificial Sequence
  Designed nucleotides based on the discovery of an optimal set barcoded oligonucleotides for multiplexed RNA sequencing.
accttctcacggcc

SEQ ID NO: 116 - DNA, 14 bp, Artificial Sequence
  Designed nucleotides based on the discovery of an optimal set barcoded oligonucleotides for multiplexed RNA sequencing.
caacgtggacctac

SEQ ID NO: 117 - DNA, 14 bp, Artificial Sequence
  Designed nucleotides based on the discovery of an optimal set barcoded oligonucleotides for multiplexed RNA sequencing.
aactgctacgcacc

SEQ ID NO: 118 - DNA, 14 bp, Artificial Sequence
  Designed nucleotides based on the discovery of an optimal set barcoded oligonucleotides for multiplexed RNA sequencing.
aacttccggcgtgc

SEQ ID NO: 119 - DNA, 14 bp, Artificial Sequence
  Designed nucleotides based on the discovery of an optimal set barcoded oligonucleotides for multiplexed RNA sequencing.
catcgtagcagagc

SEQ ID NO: 120 - DNA, 14 bp, Artificial Sequence
  Designed nucleotides based on the discovery of an optimal set barcoded oligonucleotides for multiplexed RNA sequencing.
aacaaccaagcggc

SEQ ID NO: 121 - DNA, 14 bp, Artificial Sequence
  Designed nucleotides based on the discovery of an optimal set barcoded oligonucleotides for multiplexed RNA sequencing.
taaccggccgcaac

SEQ ID NO: 122 - DNA, 14 bp, Artificial Sequence
  Designed nucleotides based on the discovery of an optimal set barcoded oligonucleotides for multiplexed RNA sequencing.
ataaccatccgaac

SEQ ID NO: 123 - DNA, 14 bp, Artificial Sequence
  Designed nucleotides based on the discovery of an optimal set barcoded oligonucleotides for multiplexed RNA sequencing.
ctagctgtgtcagc

SEQ ID NO: 124 - DNA, 14 bp, Artificial Sequence
  Designed nucleotides based on the discovery of an optimal set barcoded oligonucleotides for multiplexed RNA sequencing.
ctaaccttcattgc

SEQ ID NO: 125 - DNA, 14 bp, Artificial Sequence
  Designed nucleotides based on the discovery of an optimal set barcoded oligonucleotides for multiplexed RNA sequencing.
ctcacctaggccac

SEQ ID NO: 126 - DNA, 14 bp, Artificial Sequence
  Designed nucleotides based on the discovery of an optimal set barcoded oligonucleotides for multiplexed RNA sequencing.
tcatgcgcatgtag

SEQ ID NO: 127 - DNA, 14 bp, Artificial Sequence
  Designed nucleotides based on the discovery of an optimal set barcoded oligonucleotides for multiplexed RNA sequencing.
tcgcaattacctaa

SEQ ID NO: 128 - DNA, 14 bp, Artificial Sequence
  Designed nucleotides based on the discovery of an optimal set barcoded oligonucleotides for multiplexed RNA sequencing.
catctacgctacgc

SEQ ID NO: 129 - DNA, 14 bp, Artificial Sequence
  Designed nucleotides based on the discovery of an optimal set barcoded oligonucleotides for multiplexed RNA sequencing.
ttccaccgatgtgg

SEQ ID NO: 130 - DNA, 14 bp, Artificial Sequence
  Designed nucleotides based on the discovery of an optimal set barcoded oligonucleotides for multiplexed RNA sequencing.
tagcttctccaggc

SEQ ID NO: 131 - DNA, 14 bp, Artificial Sequence
  Designed nucleotides based on the discovery of an optimal set barcoded oligonucleotides for multiplexed RNA sequencing.
ttccaaccggacag

SEQ ID NO: 132 - DNA, 14 bp, Artificial Sequence
  Designed nucleotides based on the discovery of an optimal set barcoded oligonucleotides for multiplexed RNA sequencing.
ttgaccttgctgga

SEQ ID NO: 133 - DNA, 14 bp, Artificial Sequence
  Designed nucleotides based on the discovery of an optimal set barcoded oligonucleotides for multiplexed RNA sequencing.
accttgtccaatac

SEQ ID NO: 134 - DNA, 14 bp, Artificial Sequence
  Designed nucleotides based on the discovery of an optimal set barcoded oligonucleotides for multiplexed RNA sequencing.
cttagaattagtgg

SEQ ID NO: 135 - DNA, 14 bp, Artificial Sequence
  Designed nucleotides based on the discovery of an optimal set barcoded oligonucleotides for multiplexed RNA sequencing.
aagtgaccgacggc

SEQ ID NO: 136 - DNA, 14 bp, Artificial Sequence
  Designed nucleotides based on the discovery of an optimal set barcoded oligonucleotides for multiplexed RNA sequencing.
tcggttatcttcga

SEQ ID NO: 137 - DNA, 14 bp, Artificial Sequence
  Designed nucleotides based on the discovery of an optimal set barcoded oligonucleotides for multiplexed RNA sequencing.
ctagcttgttgtag

SEQ ID NO: 138 - DNA, 14 bp, Artificial Sequence
  Designed nucleotides based on the discovery of an optimal set barcoded oligonucleotides for multiplexed RNA sequencing.
ttaagccgaacaag

SEQ ID NO: 139 - DNA, 14 bp, Artificial Sequence
  Designed nucleotides based on the discovery of an optimal set barcoded oligonucleotides for multiplexed RNA sequencing.
ttccgatgtccggc

SEQ ID NO: 140 - DNA, 14 bp, Artificial Sequence
  Designed nucleotides based on the discovery of an optimal set barcoded oligonucleotides for multiplexed RNA sequencing.
atgaatcagccgcc

SEQ ID NO: 141 - DNA, 14 bp, Artificial Sequence
  Designed nucleotides based on the discovery of an optimal set barcoded oligonucleotides for multiplexed RNA sequencing.
tcctaacaagtgcg

SEQ ID NO: 142 - DNA, 14 bp, Artificial Sequence
  Designed nucleotides based on the discovery of an optimal set barcoded oligonucleotides for multiplexed RNA sequencing.
caatagcaacacca

SEQ ID NO: 143 - DNA, 14 bp, Artificial Sequence
  Designed nucleotides based on the discovery of an optimal set barcoded oligonucleotides for multiplexed RNA sequencing.
tatacgcgacatcg

SEQ ID NO: 144 - DNA, 14 bp, Artificial Sequence
  Designed nucleotides based on the discovery of an optimal set barcoded oligonucleotides for multiplexed RNA sequencing.
ccggatggtggtac

SEQ ID NO: 145 - DNA, 14 bp, Artificial Sequence
  Designed nucleotides based on the discovery of an optimal set barcoded oligonucleotides for multiplexed RNA sequencing.
cctacggtgttcgg

SEQ ID NO: 146 - DNA, 14 bp, Artificial Sequence
  Designed nucleotides based on the discovery of an optimal set barcoded oligonucleotides for multiplexed RNA sequencing.
ctcgcacctggtaa

SEQ ID NO: 147 - DNA, 14 bp, Artificial Sequence
  Designed nucleotides based on the discovery of an optimal set barcoded oligonucleotides for multiplexed RNA sequencing.
tcgcctgaactacc

SEQ ID NO: 148 - DNA, 14 bp, Artificial Sequence
  Designed nucleotides based on the discovery of an optimal set barcoded oligonucleotides for multiplexed RNA sequencing.
tcactcatacatag

SEQ ID NO: 149 - DNA, 14 bp, Artificial Sequence
  Designed nucleotides based on the discovery of an optimal set barcoded oligonucleotides for multiplexed RNA sequencing.
cttggtctccggca

SEQ ID NO: 150 - DNA, 14 bp, Artificial Sequence
  Designed nucleotides based on the discovery of an optimal set barcoded oligonucleotides for multiplexed RNA sequencing.
ttgattaccgtaac

SEQ ID NO: 151 - DNA, 14 bp, Artificial Sequence
  Designed nucleotides based on the discovery of an optimal set barcoded oligonucleotides for multiplexed RNA sequencing.
ttgatccgtgaagc

SEQ ID NO: 152 - DNA, 14 bp, Artificial Sequence
  Designed nucleotides based on the discovery of an optimal set barcoded oligonucleotides for multiplexed RNA sequencing.
attcttcaggaaga

SEQ ID NO: 153 - DNA, 14 bp, Artificial Sequence
  Designed nucleotides based on the discovery of an optimal set barcoded oligonucleotides for multiplexed RNA sequencing.
aagccaggtaaggc

SEQ ID NO: 154 - DNA, 14 bp, Artificial Sequence
  Designed nucleotides based on the discovery of an optimal set barcoded oligonucleotides for multiplexed RNA sequencing.
ccgcggaaggataa

SEQ ID NO: 155 - DNA, 14 bp, Artificial Sequence
  Designed nucleotides based on the discovery of an optimal set barcoded oligonucleotides for multiplexed RNA sequencing.
cataattctatgcc

SEQ ID NO: 156 - DNA, 14 bp, Artificial Sequence
  Designed nucleotides based on the discovery of an optimal set barcoded oligonucleotides for multiplexed RNA sequencing.
atgaacttggcaca

SEQ ID NO: 157 - DNA, 14 bp, Artificial Sequence
  Designed nucleotides based on the discovery of an optimal set barcoded oligonucleotides for multiplexed RNA sequencing.
cttaagcttaagag

SEQ ID NO: 158 - DNA, 14 bp, Artificial Sequence
  Designed nucleotides based on the discovery of an optimal set barcoded oligonucleotides for multiplexed RNA sequencing.
tcctcggaatgaca

SEQ ID NO: 159 - DNA, 14 bp, Artificial Sequence
  Designed nucleotides based on the discovery of an optimal set barcoded oligonucleotides for multiplexed RNA sequencing.
aatcttacctaagg

SEQ ID NO: 160 - DNA, 14 bp, Artificial Sequence
  Designed nucleotides based on the discovery of an optimal set barcoded oligonucleotides for multiplexed RNA sequencing.
accactggttgccg

SEQ ID NO: 161 - DNA, 14 bp, Artificial Sequence
  Designed nucleotides based on the discovery of an optimal set barcoded oligonucleotides for multiplexed RNA sequencing.
ctccgttctgtacc

SEQ ID NO: 162 - DNA, 14 bp, Artificial Sequence
  Designed nucleotides based on the discovery of an optimal set barcoded oligonucleotides for multiplexed RNA sequencing.
cctaactcgccaga

SEQ ID NO: 163 - DNA, 14 bp, Artificial Sequence
  Designed nucleotides based on the discovery of an optimal set barcoded oligonucleotides for multiplexed RNA sequencing.
aatagtgttcaacc

SEQ ID NO: 164 - DNA, 14 bp, Artificial Sequence
  Designed nucleotides based on the discovery of an optimal set barcoded oligonucleotides for multiplexed RNA sequencing.
ctacgagtcataga

SEQ ID NO: 165 - DNA, 14 bp, Artificial Sequence
  Designed nucleotides based on the discovery of an optimal set barcoded oligonucleotides for multiplexed RNA sequencing.
acacacgacatcgc

SEQ ID NO: 166 - DNA, 14 bp, Artificial Sequence
  Designed nucleotides based on the discovery of an optimal set barcoded oligonucleotides for multiplexed RNA sequencing.
aacttacacgttgg

SEQ ID NO: 167 - DNA, 14 bp, Artificial Sequence
  Designed nucleotides based on the discovery of an optimal set barcoded oligonucleotides for multiplexed RNA sequencing.
aacacaaccttccg

SEQ ID NO: 168 - DNA, 14 bp, Artificial Sequence
  Designed nucleotides based on the discovery of an optimal set barcoded oligonucleotides for multiplexed RNA sequencing.
ataccttaatctcc

SEQ ID NO: 169 - DNA, 14 bp, Artificial Sequence
  Designed nucleotides based on the discovery of an optimal set barcoded oligonucleotides for multiplexed RNA sequencing.
taaccgcaatagga

SEQ ID NO: 170 - DNA, 14 bp, Artificial Sequence
  Designed nucleotides based on the discovery of an optimal set barcoded oligonucleotides for multiplexed RNA sequencing.
ccgaaccatagccg

SEQ ID NO: 171 - DNA, 14 bp, Artificial Sequence
  Designed nucleotides based on the discovery of an optimal set barcoded oligonucleotides for multiplexed RNA sequencing.
ttcatataaggcgc

SEQ ID NO: 172 - DNA, 14 bp, Artificial Sequence
  Designed nucleotides based on the discovery of an optimal set barcoded oligonucleotides for multiplexed RNA sequencing.
aataccagccgcgg

SEQ ID NO: 173 - DNA, 14 bp, Artificial Sequence
  Designed nucleotides based on the discovery of an optimal set barcoded oligonucleotides for multiplexed RNA sequencing.
ctacggactaaggc

SEQ ID NO: 174 - DNA, 14 bp, Artificial Sequence
  Designed nucleotides based on the discovery of an optimal set barcoded oligonucleotides for multiplexed RNA sequencing.
taatcgcctacaga

SEQ ID NO: 175 - DNA, 14 bp, Artificial Sequence
  Designed nucleotides based on the discovery of an optimal set barcoded oligonucleotides for multiplexed RNA sequencing.
ttagcctaactcgc

SEQ ID NO: 176 - DNA, 14 bp, Artificial Sequence
  Designed nucleotides based on the discovery of an optimal set barcoded oligonucleotides for multiplexed RNA sequencing.
tagtgtggtagacc

SEQ ID NO: 177 - DNA, 14 bp, Artificial Sequence
  Designed nucleotides based on the discovery of an optimal set barcoded oligonucleotides for multiplexed RNA sequencing.
caacgccatagacc

SEQ ID NO: 178 - DNA, 14 bp, Artificial Sequence
  Designed nucleotides based on the discovery of an optimal set barcoded oligonucleotides for multiplexed RNA sequencing.
taatccatcaacgc

SEQ ID NO: 179 - DNA, 14 bp, Artificial Sequence
  Designed nucleotides based on the discovery of an optimal set barcoded oligonucleotides for multiplexed RNA sequencing.
ccggtgtgcgctaa

SEQ ID NO: 180 - DNA, 14 bp, Artificial Sequence
  Designed nucleotides based on the discovery of an optimal set barcoded oligonucleotides for multiplexed RNA sequencing.
taccggtatggtcc

SEQ ID NO: 181 - DNA, 14 bp, Artificial Sequence
  Designed nucleotides based on the discovery of an optimal set barcoded oligonucleotides for multiplexed RNA sequencing.
cacgtatagatcgc

SEQ ID NO: 182 - DNA, 14 bp, Artificial Sequence
  Designed nucleotides based on the discovery of an optimal set barcoded oligonucleotides for multiplexed RNA sequencing.
aacctaagcaatcc

SEQ ID NO: 183 - DNA, 14 bp, Artificial Sequence
  Designed nucleotides based on the discovery of an optimal set barcoded oligonucleotides for multiplexed RNA sequencing.
ctagtgttcgatcc

SEQ ID NO: 184 - DNA, 14 bp, Artificial Sequence
  Designed nucleotides based on the discovery of an optimal set barcoded oligonucleotides for multiplexed RNA sequencing.
cctacacttgagcc

SEQ ID NO: 185 - DNA, 14 bp, Artificial Sequence
  Designed nucleotides based on the discovery of an optimal set barcoded oligonucleotides for multiplexed RNA sequencing.
cagcatcatactgc

SEQ ID NO: 186 - DNA, 14 bp, Artificial Sequence
  Designed nucleotides based on the discovery of an optimal set barcoded oligonucleotides for multiplexed RNA sequencing.
aatacggagacggc

SEQ ID NO: 187 - DNA, 14 bp, Artificial Sequence
  Designed nucleotides based on the discovery of an optimal set barcoded oligonucleotides for multiplexed RNA sequencing.
attgaactccatcc

SEQ ID NO: 188 - DNA, 14 bp, Artificial Sequence
  Designed nucleotides based on the discovery of an optimal set barcoded oligonucleotides for multiplexed RNA sequencing.
aagagtcggatagg

SEQ ID NO: 189 - DNA, 14 bp, Artificial Sequence
  Designed nucleotides based on the discovery of an optimal set barcoded oligonucleotides for multiplexed RNA sequencing.
aatgcaagacaacc

SEQ ID NO: 190 - DNA, 14 bp, Artificial Sequence
  Designed nucleotides based on the discovery of an optimal set barcoded oligonucleotides for multiplexed RNA sequencing.
ttcattggaagaga

SEQ ID NO: 191 - DNA, 14 bp, Artificial Sequence
  Designed nucleotides based on the discovery of an optimal set barcoded oligonucleotides for multiplexed RNA sequencing.
ttctaggacggccg

SEQ ID NO: 192 - DNA, 14 bp, Artificial Sequence
  Designed nucleotides based on the discovery of an optimal set barcoded oligonucleotides for multiplexed RNA sequencing.
ctcaaccacgttcc

SEQ ID NO: 193 - DNA, 14 bp, Artificial Sequence
  Designed nucleotides based on the discovery of an optimal set barcoded oligonucleotides for multiplexed RNA sequencing.
ctggtctgtcttcc

SEQ ID NO: 194 - DNA, 14 bp, Artificial Sequence
  Designed nucleotides based on the discovery of an optimal set barcoded oligonucleotides for multiplexed RNA sequencing.
ttcaagtcagtgcc

SEQ ID NO: 195 - DNA, 14 bp, Artificial Sequence
  Designed nucleotides based on the discovery of an optimal set barcoded oligonucleotides for multiplexed RNA sequencing.
cacacgacagagcg

SEQ ID NO: 196 - DNA, 14 bp, Artificial Sequence
  Designed nucleotides based on the discovery of an optimal set barcoded oligonucleotides for multiplexed RNA sequencing.
cctaagcctgttcg

SEQ ID NO: 197 - DNA, 14 bp, Artificial Sequence
  Designed nucleotides based on the discovery of an optimal set barcoded oligonucleotides for multiplexed RNA sequencing.
aagctggcgcctag

SEQ ID NO: 198 - DNA, 14 bp, Artificial Sequence
  Designed nucleotides based on the discovery of an optimal set barcoded oligonucleotides for multiplexed RNA sequencing.
acgcctgttggagg

SEQ ID NO: 199 - DNA, 14 bp, Artificial Sequence
  Designed nucleotides based on the discovery of an optimal set barcoded oligonucleotides for multiplexed RNA sequencing.
tctccattatacag

SEQ ID NO: 200 - DNA, 14 bp, Artificial Sequence
  Designed nucleotides based on the discovery of an optimal set barcoded oligonucleotides for multiplexed RNA sequencing.
tccgccgcactcaa

SEQ ID NO: 201 - DNA, 14 bp, Artificial Sequence
  Designed nucleotides based on the discovery of an optimal set barcoded oligonucleotides for multiplexed RNA sequencing.
tatcgatcatctac

SEQ ID NO: 202 - DNA, 14 bp, Artificial Sequence
  Designed nucleotides based on the discovery of an optimal set barcoded oligonucleotides for multiplexed RNA sequencing.
tcttgtaccggacc

SEQ ID NO: 203 - DNA, 14 bp, Artificial Sequence
  Designed nucleotides based on the discovery of an optimal set barcoded oligonucleotides for multiplexed RNA sequencing.
aacggctgaacgac

SEQ ID NO: 204 - DNA, 14 bp, Artificial Sequence
  Designed nucleotides based on the discovery of an optimal set barcoded oligonucleotides for multiplexed RNA sequencing.
ttccgaagcctaag

SEQ ID NO: 205 - DNA, 14 bp, Artificial Sequence
  Designed nucleotides based on the discovery of an optimal set barcoded oligonucleotides for multiplexed RNA sequencing.
ttctagataggtac

SEQ ID NO: 206 - DNA, 14 bp, Artificial Sequence
  Designed nucleotides based on the discovery of an optimal set barcoded oligonucleotides for multiplexed RNA sequencing.
aattctctggcgcc

SEQ ID NO: 207 - DNA, 14 bp, Artificial Sequence
  Designed nucleotides based on the discovery of an optimal set barcoded oligonucleotides for multiplexed RNA sequencing.
atgactgagagtga

SEQ ID NO: 208 - DNA, 14 bp, Artificial Sequence
  Designed nucleotides based on the discovery of an optimal set barcoded oligonucleotides for multiplexed RNA sequencing.
taattaggtcctcg

SEQ ID NO: 209 - DNA, 14 bp, Artificial Sequence
  Designed nucleotides based on the discovery of an optimal set barcoded oligonucleotides for multiplexed RNA sequencing.
aaccgtccaagcgc

SEQ ID NO: 210 - DNA, 14 bp, Artificial Sequence
  Designed nucleotides based on the discovery of an optimal set barcoded oligonucleotides for multiplexed RNA sequencing.
acattattggccaa

SEQ ID NO: 211 - DNA, 14 bp, Artificial Sequence
  Designed nucleotides based on the discovery of an optimal set barcoded oligonucleotides for multiplexed RNA sequencing.
tccacagaaccggc

SEQ ID NO: 212 - DNA, 14 bp, Artificial Sequence
  Designed nucleotides based on the discovery of an optimal set barcoded oligonucleotides for multiplexed RNA sequencing.
tagtgatctgtagc

SEQ ID NO: 213 - DNA, 14 bp, Artificial Sequence
  Designed nucleotides based on the discovery of an optimal set barcoded oligonucleotides for multiplexed RNA sequencing.
cattcacgcttgaa

SEQ ID NO: 214 - DNA, 14 bp, Artificial Sequence
  Designed nucleotides based on the discovery of an optimal set barcoded oligonucleotides for multiplexed RNA sequencing.
cacacacgagatgc

SEQ ID NO: 215 - DNA, 14 bp, Artificial Sequence
  Designed nucleotides based on the discovery of an optimal set barcoded oligonucleotides for multiplexed RNA sequencing.
ccatgaccaccgga

SEQ ID NO: 216 - DNA, 14 bp, Artificial Sequence
  Designed nucleotides based on the discovery of an optimal set barcoded oligonucleotides for multiplexed RNA sequencing.
caacaagacctaag

SEQ ID NO: 217 - DNA, 14 bp, Artificial Sequence
  Designed nucleotides based on the discovery of an optimal set barcoded oligonucleotides for multiplexed RNA sequencing.
aaggaacctctcgg

SEQ ID NO: 218 - DNA, 14 bp, Artificial Sequence
  Designed nucleotides based on the discovery of an optimal set barcoded oligonucleotides for multiplexed RNA sequencing.
cataaccggcacaa

SEQ ID NO: 219 - DNA, 14 bp, Artificial Sequence
  Designed nucleotides based on the discovery of an optimal set barcoded oligonucleotides for multiplexed RNA sequencing.
ccaagtggtacagc

SEQ ID NO: 220 - DNA, 14 bp, Artificial Sequence
  Designed nucleotides based on the discovery of an optimal set barcoded oligonucleotides for multiplexed RNA sequencing.
ttccttaccgcgcg

SEQ ID NO: 221 - DNA, 14 bp, Artificial Sequence
  Designed nucleotides based on the discovery of an optimal set barcoded oligonucleotides for multiplexed RNA sequencing.
atactagtagtcag

SEQ ID NO: 222 - DNA, 14 bp, Artificial Sequence
  Designed nucleotides based on the discovery of an optimal set barcoded oligonucleotides for multiplexed RNA sequencing.
tatatgctcgctca

SEQ ID NO: 223 - DNA, 14 bp, Artificial Sequence
  Designed nucleotides based on the discovery of an optimal set barcoded oligonucleotides for multiplexed RNA sequencing.
tccgcgatctcaac

SEQ ID NO: 224 - DNA, 14 bp, Artificial Sequence
  Designed nucleotides based on the discovery of an optimal set barcoded oligonucleotides for multiplexed RNA sequencing.
tattaccgctcagg

SEQ ID NO: 225 - DNA, 14 bp, Artificial Sequence
  Designed nucleotides based on the discovery of an optimal set barcoded oligonucleotides for multiplexed RNA sequencing.
atcgagcagtgtac

SEQ ID NO: 226 - DNA, 14 bp, Artificial Sequence
  Designed nucleotides based on the discovery of an optimal set barcoded oligonucleotides for multiplexed RNA sequencing.
catgcgtagcgcac

SEQ ID NO: 227 - DNA, 14 bp, Artificial Sequence
  Designed nucleotides based on the discovery of an optimal set barcoded oligonucleotides for multiplexed RNA sequencing.
tccaacagtgttgg

SEQ ID NO: 228 - DNA, 14 bp, Artificial Sequence
  Designed nucleotides based on the discovery of an optimal set barcoded oligonucleotides for multiplexed RNA sequencing.
cacttggaccatcc

SEQ ID NO: 229 - DNA, 14 bp, Artificial Sequence
  ADesigned nucleotides based on the discovery of an optimal set barcoded oligonucleotides for multiplexed RNA sequencing.
ttacctggccatcc

SEQ ID NO: 230 - DNA, 14 bp, Artificial Sequence
  Designed nucleotides based on the discovery of an optimal set barcoded oligonucleotides for multiplexed RNA sequencing.
catcatgagagcga

SEQ ID NO: 231 - DNA, 14 bp, Artificial Sequence
  Designed nucleotides based on the discovery of an optimal set barcoded oligonucleotides for multiplexed RNA sequencing.
attccgaatgtaac

SEQ ID NO: 232 - DNA, 14 bp, Artificial Sequence
  Designed nucleotides based on the discovery of an optimal set barcoded oligonucleotides for multiplexed RNA sequencing.
ccggtatatccgga

SEQ ID NO: 233 - DNA, 14 bp, Artificial Sequence
  Designed nucleotides based on the discovery of an optimal set barcoded oligonucleotides for multiplexed RNA sequencing.
actgacgtggaacc

SEQ ID NO: 234 - DNA, 14 bp, Artificial Sequence
  Designed nucleotides based on the discovery of an optimal set barcoded oligonucleotides for multiplexed RNA sequencing.
ttaagctgccttaa

SEQ ID NO: 235 - DNA, 14 bp, Artificial Sequence
  Designed nucleotides based on the discovery of an optimal set barcoded oligonucleotides for multiplexed RNA sequencing.
ctccgcagttggag

SEQ ID NO: 236 - DNA, 14 bp, Artificial Sequence
  Designed nucleotides based on the discovery of an optimal set barcoded oligonucleotides for multiplexed RNA sequencing.
tcctaattctaagg

SEQ ID NO: 237 - DNA, 14 bp, Artificial Sequence
  Designed nucleotides based on the discovery of an optimal set barcoded oligonucleotides for multiplexed RNA sequencing.
tacgatcgtgatca

SEQ ID NO: 238 - DNA, 14 bp, Artificial Sequence
  Designed nucleotides based on the discovery of an optimal set barcoded oligonucleotides for multiplexed RNA sequencing.
ataaccaactagga

SEQ ID NO: 239 - DNA, 14 bp, Artificial Sequence
  Designed nucleotides based on the discovery of an optimal set barcoded oligonucleotides for multiplexed RNA sequencing.
atccttatacgtgc

SEQ ID NO: 240 - DNA, 14 bp, Artificial Sequence
  Designed nucleotides based on the discovery of an optimal set barcoded oligonucleotides for multiplexed RNA sequencing.
acttccaggcctga

SEQ ID NO: 241 - DNA, 14 bp, Artificial Sequence
  Designed nucleotides based on the discovery of an optimal set barcoded oligonucleotides for multiplexed RNA sequencing.
tctgcacatcacgc

SEQ ID NO: 242 - DNA, 14 bp, Artificial Sequence
  Designed nucleotides based on the discovery of an optimal set barcoded oligonucleotides for multiplexed RNA sequencing.
acagacgcatcacg

SEQ ID NO: 243 - DNA, 14 bp, Artificial Sequence
  Designed nucleotides based on the discovery of an optimal set barcoded oligonucleotides for multiplexed RNA sequencing.
taagttgtgtgtcc

SEQ ID NO: 244 - DNA, 14 bp, Artificial Sequence
  Designed nucleotides based on the discovery of an optimal set barcoded oligonucleotides for multiplexed RNA sequencing.
actcaacggcaagg

SEQ ID NO: 245 - DNA, 14 bp, Artificial Sequence
  Designed nucleotides based on the discovery of an optimal set barcoded oligonucleotides for multiplexed RNA sequencing.
tagcacatactcga

SEQ ID NO: 246 - DNA, 14 bp, Artificial Sequence
  Designed nucleotides based on the discovery of an optimal set barcoded oligonucleotides for multiplexed RNA sequencing.
atatcacagtcgca

SEQ ID NO: 247 - DNA, 14 bp, Artificial Sequence
  Designed nucleotides based on the discovery of an optimal set barcoded oligonucleotides for multiplexed RNA sequencing.
atatacacatgagc

SEQ ID NO: 248 - DNA, 14 bp, Artificial Sequence
  Designed nucleotides based on the discovery of an optimal set barcoded oligonucleotides for multiplexed RNA sequencing.
acgtacgcgcatgc

SEQ ID NO: 249 - DNA, 14 bp, Artificial Sequence
  Designed nucleotides based on the discovery of an optimal set barcoded oligonucleotides for multiplexed RNA sequencing.
caagccagttccgg

SEQ ID NO: 250 - DNA, 14 bp, Artificial Sequence
  Designed nucleotides based on the discovery of an optimal set barcoded oligonucleotides for multiplexed RNA sequencing.
ccgaacaaccgtga

SEQ ID NO: 251 - DNA, 14 bp, Artificial Sequence
  Designed nucleotides based on the discovery of an optimal set barcoded oligonucleotides for multiplexed RNA sequencing.
cacacacatagcga

SEQ ID NO: 252 - DNA, 14 bp, Artificial Sequence
  Designed nucleotides based on the discovery of an optimal set barcoded oligonucleotides for multiplexed RNA sequencing.
acagcgtacgcgca

SEQ ID NO: 253 - DNA, 14 bp, Artificial Sequence
  Designed nucleotides based on the discovery of an optimal set barcoded oligonucleotides for multiplexed RNA sequencing.
tcaacgctcggagc

SEQ ID NO: 254 - DNA, 14 bp, Artificial Sequence
  Designed nucleotides based on the discovery of an optimal set barcoded oligonucleotides for multiplexed RNA sequencing.
actacgatcataga

SEQ ID NO: 255 - DNA, 14 bp, Artificial Sequence
  Designed nucleotides based on the discovery of an optimal set barcoded oligonucleotides for multiplexed RNA sequencing.
caggcgtaagtgcc

SEQ ID NO: 256 - DNA, 14 bp, Artificial Sequence
  Designed nucleotides based on the discovery of an optimal set barcoded oligonucleotides for multiplexed RNA sequencing.
acatatcatgagca

SEQ ID NO: 257 - DNA, 14 bp, Artificial Sequence
  Designed nucleotides based on the discovery of an optimal set barcoded oligonucleotides for multiplexed RNA sequencing.
aactccgtacaagc

SEQ ID NO: 258 - DNA, 14 bp, Artificial Sequence
  Designed nucleotides based on the discovery of an optimal set barcoded oligonucleotides for multiplexed RNA sequencing.
ctgtcgagtagcag

SEQ ID NO: 259 - DNA, 14 bp, Artificial Sequence
  Designed nucleotides based on the discovery of an optimal set barcoded oligonucleotides for multiplexed RNA sequencing.
tagacgatagtaca

SEQ ID NO: 260 - DNA, 14 bp, Artificial Sequence
  Designed nucleotides based on the discovery of an optimal set barcoded oligonucleotides for multiplexed RNA sequencing.
catgctcacgcagc

SEQ ID NO: 261 - DNA, 14 bp, Artificial Sequence
  Designed nucleotides based on the discovery of an optimal set barcoded oligonucleotides for multiplexed RNA sequencing.
atcacgagcgacgc

SEQ ID NO: 262 - DNA, 14 bp, Artificial Sequence
  Designed nucleotides based on the discovery of an optimal set barcoded oligonucleotides for multiplexed RNA sequencing.
cagcctggcttagg

SEQ ID NO: 263 - DNA, 14 bp, Artificial Sequence
  Designed nucleotides based on the discovery of an optimal set barcoded oligonucleotides for multiplexed RNA sequencing.
ccagaggttgccag

SEQ ID NO: 264 - DNA, 14 bp, Artificial Sequence
  Designed nucleotides based on the discovery of an optimal set barcoded oligonucleotides for multiplexed RNA sequencing.
tactatgcacgacg

SEQ ID NO: 265 - DNA, 14 bp, Artificial Sequence
  Designed nucleotides based on the discovery of an optimal set barcoded oligonucleotides for multiplexed RNA sequencing.
acgctgacgtgcga

SEQ ID NO: 266 - DNA, 14 bp, Artificial Sequence
  Designed nucleotides based on the discovery of an optimal set barcoded oligonucleotides for multiplexed RNA sequencing.
cagcacatgtacag

SEQ ID NO: 267 - DNA, 14 bp, Artificial Sequence
  Designed nucleotides based on the discovery of an optimal set barcoded oligonucleotides for multiplexed RNA sequencing.
acacgcatcatacg

SEQ ID NO: 268 - DNA, 14 bp, Artificial Sequence
  Designed nucleotides based on the discovery of an optimal set barcoded oligonucleotides for multiplexed RNA sequencing.
ctgtacacgtctac

SEQ ID NO: 269 - DNA, 14 bp, Artificial Sequence
  Designed nucleotides based on the discovery of an optimal set barcoded oligonucleotides for multiplexed RNA sequencing.
cacacagtgagtac

SEQ ID NO: 270 - DNA, 14 bp, Artificial Sequence
  Designed nucleotides based on the discovery of an optimal set barcoded oligonucleotides for multiplexed RNA sequencing.
tagcacgcgatgag

SEQ ID NO: 271 - DNA, 14 bp, Artificial Sequence
  Designed nucleotides based on the discovery of an optimal set barcoded oligonucleotides for multiplexed RNA sequencing.
tccaataggtccag

SEQ ID NO: 272 - DNA, 14 bp, Artificial Sequence
  Designed nucleotides based on the discovery of an optimal set barcoded oligonucleotides for multiplexed RNA sequencing.
ctcctgcggtccaa

SEQ ID NO: 273 - DNA, 14 bp, Artificial Sequence
  Designed nucleotides based on the discovery of an optimal set barcoded oligonucleotides for multiplexed RNA sequencing.
atggcaacggctca

SEQ ID NO: 274 - DNA, 14 bp, Artificial Sequence
  Designed nucleotides based on the discovery of an optimal set barcoded oligonucleotides for multiplexed RNA sequencing.
ccgaggcttcctag

SEQ ID NO: 275 - DNA, 14 bp, Artificial Sequence
  Designed nucleotides based on the discovery of an optimal set barcoded oligonucleotides for multiplexed RNA sequencing.
acgacagtagtcgc

SEQ ID NO: 276 - DNA, 14 bp, Artificial Sequence
  Designed nucleotides based on the discovery of an optimal set barcoded oligonucleotides for multiplexed RNA sequencing.
ttataactggcaac

SEQ ID NO: 277 - DNA, 14 bp, Artificial Sequence
  Designed nucleotides based on the discovery of an optimal set barcoded oligonucleotides for multiplexed RNA sequencing.
actctgagtatcag

SEQ ID NO: 278 - DNA, 14 bp, Artificial Sequence
  Designed nucleotides based on the discovery of an optimal set barcoded oligonucleotides for multiplexed RNA sequencing.
ttatgtcttaacgg

SEQ ID NO: 279 - DNA, 14 bp, Artificial Sequence
  Designed nucleotides based on the discovery of an optimal set barcoded oligonucleotides for multiplexed RNA sequencing.
atgcgactcgagac

SEQ ID NO: 280 - DNA, 14 bp, Artificial Sequence
  Designed nucleotides based on the discovery of an optimal set barcoded oligonucleotides for multiplexed RNA sequencing.
cctcaattccggcg

SEQ ID NO: 281 - DNA, 14 bp, Artificial Sequence
  Designed nucleotides based on the discovery of an optimal set barcoded oligonucleotides for multiplexed RNA sequencing.
aatggttggcagag

SEQ ID NO: 282 - DNA, 14 bp, Artificial Sequence
  Designed nucleotides based on the discovery of an optimal set barcoded oligonucleotides for multiplexed RNA sequencing.
atgcaagcgattcc

SEQ ID NO: 283 - DNA, 14 bp, Artificial Sequence
  Designed nucleotides based on the discovery of an optimal set barcoded oligonucleotides for multiplexed RNA sequencing.
tattgagtccgaga

SEQ ID NO: 284 - DNA, 14 bp, Artificial Sequence
  Designed nucleotides based on the discovery of an optimal set barcoded oligonucleotides for multiplexed RNA sequencing.
ccgcgtattgcaac

SEQ ID NO: 285 - DNA, 14 bp, Artificial Sequence
  Designed nucleotides based on the discovery of an optimal set barcoded oligonucleotides for multiplexed RNA sequencing.
tcattctacttggc

SEQ ID NO: 286 - DNA, 14 bp, Artificial Sequence
  Designed nucleotides based on the discovery of an optimal set barcoded oligonucleotides for multiplexed RNA sequencing.
atcatcgcgcgacg

SEQ ID NO: 287 - DNA, 14 bp, Artificial Sequence
  Designed nucleotides based on the discovery of an optimal set barcoded oligonucleotides for multiplexed RNA sequencing.
cagagacactcgca

SEQ ID NO: 288 - DNA, 14 bp, Artificial Sequence
  Designed nucleotides based on the discovery of an optimal set barcoded oligonucleotides for multiplexed RNA sequencing.
aagaagagtggacc

SEQ ID NO: 289 - DNA, 14 bp, Artificial Sequence
  Designed nucleotides based on the discovery of an optimal set barcoded oligonucleotides for multiplexed RNA sequencing.
acacacgctgcgac

SEQ ID NO: 290 - DNA, 14 bp, Artificial Sequence
  Designed nucleotides based on the discovery of an optimal set barcoded oligonucleotides for multiplexed RNA sequencing.
tccgcaagtcatag

SEQ ID NO: 291 - DNA, 14 bp, Artificial Sequence
  Designed nucleotides based on the discovery of an optimal set barcoded oligonucleotides for multiplexed RNA sequencing.
aatgtcctcagaac

SEQ ID NO: 292 - DNA, 14 bp, Artificial Sequence
  Designed nucleotides based on the discovery of an optimal set barcoded oligonucleotides for multiplexed RNA sequencing.
ccgagtgtaagtcc

SEQ ID NO: 293 - DNA, 14 bp, Artificial Sequence
  Designed nucleotides based on the discovery of an optimal set barcoded oligonucleotides for multiplexed RNA sequencing.
acacgtactcatac

SEQ ID NO: 294 - DNA, 14 bp, Artificial Sequence
  Designed nucleotides based on the discovery of an optimal set barcoded oligonucleotides for multiplexed RNA sequencing.
ttctctccggttca

SEQ ID NO: 295 - DNA, 14 bp, Artificial Sequence
  Designed nucleotides based on the discovery of an optimal set barcoded oligonucleotides for multiplexed RNA sequencing.
aatccgtgtcttaa

SEQ ID NO: 296 - DNA, 14 bp, Artificial Sequence
  Designed nucleotides based on the discovery of an optimal set barcoded oligonucleotides for multiplexed RNA sequencing.
ccttgccagagtgg

SEQ ID NO: 297 - DNA, 14 bp, Artificial Sequence
  Designed nucleotides based on the discovery of an optimal set barcoded oligonucleotides for multiplexed RNA sequencing.
cttactgacaagcc

SEQ ID NO: 298 - DNA, 14 bp, Artificial Sequence
  Designed nucleotides based on the discovery of an optimal set barcoded oligonucleotides for multiplexed RNA sequencing.
tcctccgatgccag

SEQ ID NO: 299 - DNA, 14 bp, Artificial Sequence
  Designed nucleotides based on the discovery of an optimal set barcoded oligonucleotides for multiplexed RNA sequencing.
ttcttctccagaag

SEQ ID NO: 300 - DNA, 14 bp, Artificial Sequence
  Designed nucleotides based on the discovery of an optimal set barcoded oligonucleotides for multiplexed RNA sequencing.
cacctgttggttaa

SEQ ID NO: 301 - DNA, 14 bp, Artificial Sequence
  Designed nucleotides based on the discovery of an optimal set barcoded oligonucleotides for multiplexed RNA sequencing.
ctcagtcacacgag

SEQ ID NO: 302 - DNA, 14 bp, Artificial Sequence
  Designed nucleotides based on the discovery of an optimal set barcoded oligonucleotides for multiplexed RNA sequencing.
ccttatctacgacc

SEQ ID NO: 303 - DNA, 14 bp, Artificial Sequence
  Designed nucleotides based on the discovery of an optimal set barcoded oligonucleotides for multiplexed RNA sequencing.
aaccgaacgcttaa

SEQ ID NO: 304 - DNA, 14 bp, Artificial Sequence
  Designed nucleotides based on the discovery of an optimal set barcoded oligonucleotides for multiplexed RNA sequencing.
acaggtaggaagga

SEQ ID NO: 305 - DNA, 14 bp, Artificial Sequence
  Designed nucleotides based on the discovery of an optimal set barcoded oligonucleotides for multiplexed RNA sequencing.
cagcatagtctgac

SEQ ID NO: 306 - DNA, 14 bp, Artificial Sequence
  Designed nucleotides based on the discovery of an optimal set barcoded oligonucleotides for multiplexed RNA sequencing.
attcggtccgatgc

SEQ ID NO: 307 - DNA, 14 bp, Artificial Sequence
  Designed nucleotides based on the discovery of an optimal set barcoded oligonucleotides for multiplexed RNA sequencing.
actaacctggccgg

SEQ ID NO: 308 - DNA, 14 bp, Artificial Sequence
  Designed nucleotides based on the discovery of an optimal set barcoded oligonucleotides for multiplexed RNA sequencing.
cattcctctagccg

SEQ ID NO: 309 - DNA, 14 bp, Artificial Sequence
  Designed nucleotides based on the discovery of an optimal set barcoded oligonucleotides for multiplexed RNA sequencing.
cttaatccatatcc

SEQ ID NO: 310 - DNA, 14 bp, Artificial Sequence
  Designed nucleotides based on the discovery of an optimal set barcoded oligonucleotides for multiplexed RNA sequencing.
aacaagaggtgtgg

SEQ ID NO: 311 - DNA, 14 bp, Artificial Sequence
  Designed nucleotides based on the discovery of an optimal set barcoded oligonucleotides for multiplexed RNA sequencing.
tatcatatgagtcg

SEQ ID NO: 312 - DNA, 14 bp, Artificial Sequence
  Designed nucleotides based on the discovery of an optimal set barcoded oligonucleotides for multiplexed RNA sequencing.
acgagtatgtatgc

SEQ ID NO: 313 - DNA, 14 bp, Artificial Sequence
  Designed nucleotides based on the discovery of an optimal set barcoded oligonucleotides for multiplexed RNA sequencing.
tcgcggattaggca

SEQ ID NO: 314 - DNA, 14 bp, Artificial Sequence
  Designed nucleotides based on the discovery of an optimal set barcoded oligonucleotides for multiplexed RNA sequencing.
ctactgtgacacac

SEQ ID NO: 315 - DNA, 14 bp, Artificial Sequence
  Designed nucleotides based on the discovery of an optimal set barcoded oligonucleotides for multiplexed RNA sequencing.
caaccgtgtgaacc

SEQ ID NO: 316 - DNA, 14 bp, Artificial Sequence
  Designed nucleotides based on the discovery of an optimal set barcoded oligonucleotides for multiplexed RNA sequencing.
cagcactagctgca

SEQ ID NO: 317 - DNA, 14 bp, Artificial Sequence
  Designed nucleotides based on the discovery of an optimal set barcoded oligonucleotides for multiplexed RNA sequencing.
acacgacatgcacg

SEQ ID NO: 318 - DNA, 14 bp, Artificial Sequence
  Designed nucleotides based on the discovery of an optimal set barcoded oligonucleotides for multiplexed RNA sequencing.
actcatgtcagaca

SEQ ID NO: 319 - DNA, 14 bp, Artificial Sequence
  Designed nucleotides based on the discovery of an optimal set barcoded oligonucleotides for multiplexed RNA sequencing.
acactatacacgag

SEQ ID NO: 320 - DNA, 14 bp, Artificial Sequence
  Designed nucleotides based on the discovery of an optimal set barcoded oligonucleotides for multiplexed RNA sequencing.
acttgagcaaccgg

SEQ ID NO: 321 - DNA, 14 bp, Artificial Sequence
  Designed nucleotides based on the discovery of an optimal set barcoded oligonucleotides for multiplexed RNA sequencing.
ttagacgccggtaa

SEQ ID NO: 322 - DNA, 14 bp, Artificial Sequence
  Designed nucleotides based on the discovery of an optimal set barcoded oligonucleotides for multiplexed RNA sequencing.
ctgtgtaggattaa

SEQ ID NO: 323 - DNA, 14 bp, Artificial Sequence
  Designed nucleotides based on the discovery of an optimal set barcoded oligonucleotides for multiplexed RNA sequencing.
cacgcatcagtcag

SEQ ID NO: 324 - DNA, 14 bp, Artificial Sequence
  Designed nucleotides based on the discovery of an optimal set barcoded oligonucleotides for multiplexed RNA sequencing.
caaccattggcaag

SEQ ID NO: 325 - DNA, 14 bp, Artificial Sequence
  Designed nucleotides based on the discovery of an optimal set barcoded oligonucleotides for multiplexed RNA sequencing.
tcatctgcgcacac

SEQ ID NO: 326 - DNA, 14 bp, Artificial Sequence
  Designed nucleotides based on the discovery of an optimal set barcoded oligonucleotides for multiplexed RNA sequencing.
taggcctctgatgg

SEQ ID NO: 327 - DNA, 14 bp, Artificial Sequence
  Designed nucleotides based on the discovery of an optimal set barcoded oligonucleotides for multiplexed RNA sequencing.
tagcatgtcagcac

SEQ ID NO: 328 - DNA, 14 bp, Artificial Sequence
  Designed nucleotides based on the discovery of an optimal set barcoded oligonucleotides for multiplexed RNA sequencing.
tcctggacgatgcc

SEQ ID NO: 329 - DNA, 14 bp, Artificial Sequence
  Designed nucleotides based on the discovery of an optimal set barcoded oligonucleotides for multiplexed RNA sequencing.
atatcacatgacag

SEQ ID NO: 330 - DNA, 14 bp, Artificial Sequence
  Designed nucleotides based on the discovery of an optimal set barcoded oligonucleotides for multiplexed RNA sequencing.
actacgtcgacacg

SEQ ID NO: 331 - DNA, 14 bp, Artificial Sequence
  Designed nucleotides based on the discovery of an optimal set barcoded oligonucleotides for multiplexed RNA sequencing.
aatccaactcgaag

SEQ ID NO: 332 - DNA, 14 bp, Artificial Sequence
  Designed nucleotides based on the discovery of an optimal set barcoded oligonucleotides for multiplexed RNA sequencing.
aaggatgccagtgg

SEQ ID NO: 333 - DNA, 14 bp, Artificial Sequence
  Designed nucleotides based on the discovery of an optimal set barcoded oligonucleotides for multiplexed RNA sequencing.
accgccttggctag

SEQ ID NO: 334 - DNA, 14 bp, Artificial Sequence
  Designed nucleotides based on the discovery of an optimal set barcoded oligonucleotides for multiplexed RNA sequencing.
ctgtgattcggacc

SEQ ID NO: 335 - DNA, 14 bp, Artificial Sequence
  Designed nucleotides based on the discovery of an optimal set barcoded oligonucleotides for multiplexed RNA sequencing.
catgagacgtcgcg

SEQ ID NO: 336 - DNA, 14 bp, Artificial Sequence
  Designed nucleotides based on the discovery of an optimal set barcoded oligonucleotides for multiplexed RNA sequencing.
aatccgcctaatgg

SEQ ID NO: 337 - DNA, 14 bp, Artificial Sequence
  Designed nucleotides based on the discovery of an optimal set barcoded oligonucleotides for multiplexed RNA sequencing.
aatgccgtattgac

SEQ ID NO: 338 - DNA, 14 bp, Artificial Sequence
  Designed nucleotides based on the discovery of an optimal set barcoded oligonucleotides for multiplexed RNA sequencing.
ctccgaagaaccgg

SEQ ID NO: 339 - DNA, 14 bp, Artificial Sequence
  Designed nucleotides based on the discovery of an optimal set barcoded oligonucleotides for multiplexed RNA sequencing.
actgattagaatcg

SEQ ID NO: 340 - DNA, 14 bp, Artificial Sequence
  Designed nucleotides based on the discovery of an optimal set barcoded oligonucleotides for multiplexed RNA sequencing.
actcgccaccgaag

SEQ ID NO: 341 - DNA, 14 bp, Artificial Sequence
  Designed nucleotides based on the discovery of an optimal set barcoded oligonucleotides for multiplexed RNA sequencing.
ccggtattgttaca

SEQ ID NO: 342 - DNA, 14 bp, Artificial Sequence
  Designed nucleotides based on the discovery of an optimal set barcoded oligonucleotides for multiplexed RNA sequencing.
ttggtgagaaccgc

SEQ ID NO: 343 - DNA, 14 bp, Artificial Sequence
  Designed nucleotides based on the discovery of an optimal set barcoded oligonucleotides for multiplexed RNA sequencing.
cagatctcagacac

SEQ ID NO: 344 - DNA, 14 bp, Artificial Sequence
  Designed nucleotides based on the discovery of an optimal set barcoded oligonucleotides for multiplexed RNA sequencing.
ccttaacacggaga

SEQ ID NO: 345 - DNA, 14 bp, Artificial Sequence
  Designed nucleotides based on the discovery of an optimal set barcoded oligonucleotides for multiplexed RNA sequencing.
atgtctagtatagc

SEQ ID NO: 346 - DNA, 14 bp, Artificial Sequence
  Designed nucleotides based on the discovery of an optimal set barcoded oligonucleotides for multiplexed RNA sequencing.
ccagaggtaatgga

SEQ ID NO: 347 - DNA, 14 bp, Artificial Sequence
  Designed nucleotides based on the discovery of an optimal set barcoded oligonucleotides for multiplexed RNA sequencing.
ctgtcacacagtca

SEQ ID NO: 348 - DNA, 14 bp, Artificial Sequence
  Designed nucleotides based on the discovery of an optimal set barcoded oligonucleotides for multiplexed RNA sequencing.
tcactgagtgctga

SEQ ID NO: 349 - DNA, 14 bp, Artificial Sequence
  Designed nucleotides based on the discovery of an optimal set barcoded oligonucleotides for multiplexed RNA sequencing.
tcctatggtaggag

SEQ ID NO: 350 - DNA, 14 bp, Artificial Sequence
  Designed nucleotides based on the discovery of an optimal set barcoded oligonucleotides for multiplexed RNA sequencing.
aaggaacaatacac

SEQ ID NO: 351 - DNA, 14 bp, Artificial Sequence
  Designed nucleotides based on the discovery of an optimal set barcoded oligonucleotides for multiplexed RNA sequencing.
taagccggaattag

SEQ ID NO: 352 - DNA, 14 bp, Artificial Sequence
  Designed nucleotides based on the discovery of an optimal set barcoded oligonucleotides for multiplexed RNA sequencing.
ataatgctccagaa

SEQ ID NO: 353 - DNA, 14 bp, Artificial Sequence
  Designed nucleotides based on the discovery of an optimal set barcoded oligonucleotides for multiplexed RNA sequencing.
attcttgcgaagcg

SEQ ID NO: 354 - DNA, 14 bp, Artificial Sequence
  Designed nucleotides based on the discovery of an optimal set barcoded oligonucleotides for multiplexed RNA sequencing.
acatgctatactac

SEQ ID NO: 355 - DNA, 14 bp, Artificial Sequence
  Designed nucleotides based on the discovery of an optimal set barcoded oligonucleotides for multiplexed RNA sequencing.
aagccaacagttgg

SEQ ID NO: 356 - DNA, 14 bp, Artificial Sequence
  Designed nucleotides based on the discovery of an optimal set barcoded oligonucleotides for multiplexed RNA sequencing.
tatgagcacacgac

SEQ ID NO: 357 - DNA, 14 bp, Artificial Sequence
  Designed nucleotides based on the discovery of an optimal set barcoded oligonucleotides for multiplexed RNA sequencing.
cactagatatcaca

SEQ ID NO: 358 - DNA, 14 bp, Artificial Sequence
  Designed nucleotides based on the discovery of an optimal set barcoded oligonucleotides for multiplexed RNA sequencing.
tcaattaaggcacg

SEQ ID NO: 359 - DNA, 14 bp, Artificial Sequence
  Designed nucleotides based on the discovery of an optimal set barcoded oligonucleotides for multiplexed RNA sequencing.
tcctcctcttgtga

SEQ ID NO: 360 - DNA, 14 bp, Artificial Sequence
  Designed nucleotides based on the discovery of an optimal set barcoded oligonucleotides for multiplexed RNA sequencing.
tactactatgtgac

SEQ ID NO: 361 - DNA, 14 bp, Artificial Sequence
  Designed nucleotides based on the discovery of an optimal set barcoded oligonucleotides for multiplexed RNA sequencing.
tatcttccacctga

SEQ ID NO: 362 - DNA, 14 bp, Artificial Sequence
  Designed nucleotides based on the discovery of an optimal set barcoded oligonucleotides for multiplexed RNA sequencing.
cacttaccttacaa

SEQ ID NO: 363 - DNA, 14 bp, Artificial Sequence
  Designed nucleotides based on the discovery of an optimal set barcoded oligonucleotides for multiplexed RNA sequencing.
tctagcatcgacga

SEQ ID NO: 364 - DNA, 14 bp, Artificial Sequence
  Designed nucleotides based on the discovery of an optimal set barcoded oligonucleotides for multiplexed RNA sequencing.
acacgagcacgagc

SEQ ID NO: 365 - DNA, 14 bp, Artificial Sequence
  Designed nucleotides based on the discovery of an optimal set barcoded oligonucleotides for multiplexed RNA sequencing.
acaaggcaacagcc

SEQ ID NO: 366 - DNA, 14 bp, Artificial Sequence
  Designed nucleotides based on the discovery of an optimal set barcoded oligonucleotides for multiplexed RNA sequencing.
cactacgcgctcac

SEQ ID NO: 367 - DNA, 14 bp, Artificial Sequence
  Designed nucleotides based on the discovery of an optimal set barcoded oligonucleotides for multiplexed RNA sequencing.
taaccgtctctggc

SEQ ID NO: 368 - DNA, 14 bp, Artificial Sequence
  Designed nucleotides based on the discovery of an optimal set barcoded oligonucleotides for multiplexed RNA sequencing.
ccaactcgccagga

SEQ ID NO: 369 - DNA, 14 bp, Artificial Sequence
  Designed nucleotides based on the discovery of an optimal set barcoded oligonucleotides for multiplexed RNA sequencing.
cttcggtacactaa

SEQ ID NO: 370 - DNA, 14 bp, Artificial Sequence
  Designed nucleotides based on the discovery of an optimal set barcoded oligonucleotides for multiplexed RNA sequencing.
ccacaccttcaggc

SEQ ID NO: 371 - DNA, 14 bp, Artificial Sequence
  Designed nucleotides based on the discovery of an optimal set barcoded oligonucleotides for multiplexed RNA sequencing.
ccacttcttgttgg

SEQ ID NO: 372 - DNA, 14 bp, Artificial Sequence
  Designed nucleotides based on the discovery of an optimal set barcoded oligonucleotides for multiplexed RNA sequencing.
ctagtcttggtagg

SEQ ID NO: 373 - DNA, 14 bp, Artificial Sequence
  Designed nucleotides based on the discovery of an optimal set barcoded oligonucleotides for multiplexed RNA sequencing.
tcagaatcctcgcc

SEQ ID NO: 374 - DNA, 14 bp, Artificial Sequence
  Designed nucleotides based on the discovery of an optimal set barcoded oligonucleotides for multiplexed RNA sequencing.
ctaggagcttgaac

SEQ ID NO: 375 - DNA, 14 bp, Artificial Sequence
  Designed nucleotides based on the discovery of an optimal set barcoded oligonucleotides for multiplexed RNA sequencing.
ccggcgcaattcag

SEQ ID NO: 376 - DNA, 14 bp, Artificial Sequence
  Designed nucleotides based on the discovery of an optimal set barcoded oligonucleotides for multiplexed RNA sequencing.
accggtcgttctgc

SEQ ID NO: 377 - DNA, 14 bp, Artificial Sequence
  Designed nucleotides based on the discovery of an optimal set barcoded oligonucleotides for multiplexed RNA sequencing.
cattaaggtatcgc

SEQ ID NO: 378 - DNA, 14 bp, Artificial Sequence
  Designed nucleotides based on the discovery of an optimal set barcoded oligonucleotides for multiplexed RNA sequencing.
cttggagtggcaca

SEQ ID NO: 379 - DNA, 14 bp, Artificial Sequence
  Designed nucleotides based on the discovery of an optimal set barcoded oligonucleotides for multiplexed RNA sequencing.
aaccttctagagaa

SEQ ID NO: 380 - DNA, 14 bp, Artificial Sequence
  Designed nucleotides based on the discovery of an optimal set barcoded oligonucleotides for multiplexed RNA sequencing.
catggaatgtgtaa

SEQ ID NO: 381 - DNA, 14 bp, Artificial Sequence
  Designed nucleotides based on the discovery of an optimal set barcoded oligonucleotides for multiplexed RNA sequencing.
ctgttcggaggtca

SEQ ID NO: 382 - DNA, 14 bp, Artificial Sequence
  Designed nucleotides based on the discovery of an optimal set barcoded oligonucleotides for multiplexed RNA sequencing.
atcgtgtgtggcca

SEQ ID NO: 383 - DNA, 14 bp, Artificial Sequence
  Designed nucleotides based on the discovery of an optimal set barcoded oligonucleotides for multiplexed RNA sequencing.
ttgtcggttgaggc

SEQ ID NO: 384 - DNA, 14 bp, Artificial Sequence
  Designed nucleotides based on the discovery of an optimal set barcoded oligonucleotides for multiplexed RNA sequencing.
ctggcgttgagtcg

SEQ ID NO: 385 - DNA, 14 bp, Artificial Sequence
  Designed nucleotides based on the discovery of an optimal set barcoded oligonucleotides for multiplexed RNA sequencing.
aatgaccacgaccg

SEQ ID NO: 386 - DNA, 14 bp, Artificial Sequence
  Designed nucleotides based on the discovery of an optimal set barcoded oligonucleotides for multiplexed RNA sequencing.
atgtctacagacga

SEQ ID NO: 387 - DNA, 14 bp, Artificial Sequence
  Designed nucleotides based on the discovery of an optimal set barcoded oligonucleotides for multiplexed RNA sequencing.
tctccgaggtaggc

SEQ ID NO: 388 - DNA, 14 bp, Artificial Sequence
  Designed nucleotides based on the discovery of an optimal set barcoded oligonucleotides for multiplexed RNA sequencing.
acgacgactgacag

SEQ ID NO: 389 - DNA, 14 bp, Artificial Sequence
  Designed nucleotides based on the discovery of an optimal set barcoded oligonucleotides for multiplexed RNA sequencing.
actatgactgcgca

SEQ ID NO: 390 - DNA, 14 bp, Artificial Sequence
  Designed nucleotides based on the discovery of an optimal set barcoded oligonucleotides for multiplexed RNA sequencing.
ccgccttctaacac

SEQ ID NO: 391 - DNA, 14 bp, Artificial Sequence
  Designed nucleotides based on the discovery of an optimal set barcoded oligonucleotides for multiplexed RNA sequencing.
cacacgcagtatca

SEQ ID NO: 392 - DNA, 14 bp, Artificial Sequence
  Designed nucleotides based on the discovery of an optimal set barcoded oligonucleotides for multiplexed RNA sequencing.
ccagcttaggaaga

SEQ ID NO: 393 - DNA, 14 bp, Artificial Sequence
  Designed nucleotides based on the discovery of an optimal set barcoded oligonucleotides for multiplexed RNA sequencing.
acacgatctgacga

SEQ ID NO: 394 - DNA, 14 bp, Artificial Sequence
  Designed nucleotides based on the discovery of an optimal set barcoded oligonucleotides for multiplexed RNA sequencing.
aaggcaagttgtga

SEQ ID NO: 395 - DNA, 14 bp, Artificial Sequence
  Designed nucleotides based on the discovery of an optimal set barcoded oligonucleotides for multiplexed RNA sequencing.
aactcttcggtagg

SEQ ID NO: 396 - DNA, 14 bp, Artificial Sequence
  Designed nucleotides based on the discovery of an optimal set barcoded oligonucleotides for multiplexed RNA sequencing.
ccgtccttcttcac

SEQ ID NO: 397 - DNA, 14 bp, Artificial Sequence
  Designed nucleotides based on the discovery of an optimal set barcoded oligonucleotides for multiplexed RNA sequencing.
cttggttggttccg

SEQ ID NO: 398 - DNA, 14 bp, Artificial Sequence
  Designed nucleotides based on the discovery of an optimal set barcoded oligonucleotides for multiplexed RNA sequencing.
taccttggagttgg

SEQ ID NO: 399 - DNA, 14 bp, Artificial Sequence
  Designed nucleotides based on the discovery of an optimal set barcoded oligonucleotides for multiplexed RNA sequencing.
accggcggtatagg

SEQ ID NO: 400 - DNA, 14 bp, Artificial Sequence
  Designed nucleotides based on the discovery of an optimal set barcoded oligonucleotides for multiplexed RNA sequencing.
cacgacgactcaga

SEQ ID NO: 401 - DNA, 14 bp, Artificial Sequence
  Designed nucleotides based on the discovery of an optimal set barcoded oligonucleotides for multiplexed RNA sequencing.
tcttgtgaggttcg

SEQ ID NO: 402 - DNA, 14 bp, Artificial Sequence
  Designed nucleotides based on the discovery of an optimal set barcoded oligonucleotides for multiplexed RNA sequencing.
aacgcttccggtcc

SEQ ID NO: 403 - DNA, 14 bp, Artificial Sequence
  Designed nucleotides based on the discovery of an optimal set barcoded oligonucleotides for multiplexed RNA sequencing.
ctactgcatagtag

SEQ ID NO: 404 - DNA, 14 bp, Artificial Sequence
  Designed nucleotides based on the discovery of an optimal set barcoded oligonucleotides for multiplexed RNA sequencing.
acacagcacgatag

SEQ ID NO: 405 - DNA, 14 bp, Artificial Sequence
  Designed nucleotides based on the discovery of an optimal set barcoded oligonucleotides for multiplexed RNA sequencing.
cctacaggtcgagg

SEQ ID NO: 406 - DNA, 14 bp, Artificial Sequence
  Designed nucleotides based on the discovery of an optimal set barcoded oligonucleotides for multiplexed RNA sequencing.
atgcacgatcgagc

SEQ ID NO: 407 - DNA, 14 bp, Artificial Sequence
  Designed nucleotides based on the discovery of an optimal set barcoded oligonucleotides for multiplexed RNA sequencing.
cttgataccggcgc

SEQ ID NO: 408 - DNA, 14 bp, Artificial Sequence
  Designed nucleotides based on the discovery of an optimal set barcoded oligonucleotides for multiplexed RNA sequencing.
cctcggttgaagcc

SEQ ID NO: 409 - DNA, 14 bp, Artificial Sequence
  Designed nucleotides based on the discovery of an optimal set barcoded oligonucleotides for multiplexed RNA sequencing.
caacaccggcttgg

SEQ ID NO: 410 - DNA, 14 bp, Artificial Sequence
  Designed nucleotides based on the discovery of an optimal set barcoded oligonucleotides for multiplexed RNA sequencing.
ctctggaccgatca

SEQ ID NO: 411 - DNA, 14 bp, Artificial Sequence
  Designed nucleotides based on the discovery of an optimal set barcoded oligonucleotides for multiplexed RNA sequencing.
ccgtgaaccttgcg

SEQ ID NO: 412 - DNA, 14 bp, Artificial Sequence
  Designed nucleotides based on the discovery of an optimal set barcoded oligonucleotides for multiplexed RNA sequencing.
tcatgctcgagaca

SEQ ID NO: 413 - DNA, 14 bp, Artificial Sequence
  Designed nucleotides based on the discovery of an optimal set barcoded oligonucleotides for multiplexed RNA sequencing.
accggaacctatgg

SEQ ID NO: 414 - DNA, 14 bp, Artificial Sequence
  Designed nucleotides based on the discovery of an optimal set barcoded oligonucleotides for multiplexed RNA sequencing.
ttccaaggttgaca

SEQ ID NO: 415 - DNA, 14 bp, Artificial Sequence
  Designed nucleotides based on the discovery of an optimal set barcoded oligonucleotides for multiplexed RNA sequencing.
acgagagtgctgag

SEQ ID NO: 416 - DNA, 14 bp, Artificial Sequence
  Designed nucleotides based on the discovery of an optimal set barcoded oligonucleotides for multiplexed RNA sequencing.
ctcaaccggacgga

SEQ ID NO: 417 - DNA, 14 bp, Artificial Sequence
  Designed nucleotides based on the discovery of an optimal set barcoded oligonucleotides for multiplexed RNA sequencing.
aatcaagtgtgaac

SEQ ID NO: 418 - DNA, 14 bp, Artificial Sequence
  Designed nucleotides based on the discovery of an optimal set barcoded oligonucleotides for multiplexed RNA sequencing.
catacggcctctaa

SEQ ID NO: 419 - DNA, 14 bp, Artificial Sequence
  Designed nucleotides based on the discovery of an optimal set barcoded oligonucleotides for multiplexed RNA sequencing.
actagagcgtgtga

SEQ ID NO: 420 - DNA, 14 bp, Artificial Sequence
  Designed nucleotides based on the discovery of an optimal set barcoded oligonucleotides for multiplexed RNA sequencing.
tcagagtagtgcag

SEQ ID NO: 421 - DNA, 14 bp, Artificial Sequence
  Designed nucleotides based on the discovery of an optimal set barcoded oligonucleotides for multiplexed RNA sequencing.
caacaaccttgagg

SEQ ID NO: 422 - DNA, 14 bp, Artificial Sequence
  Designed nucleotides based on the discovery of an optimal set barcoded oligonucleotides for multiplexed RNA sequencing.
acaattagccttgg

SEQ ID NO: 423 - DNA, 14 bp, Artificial Sequence
  Designed nucleotides based on the discovery of an optimal set barcoded oligonucleotides for multiplexed RNA sequencing.
tcactatgcgatac

SEQ ID NO: 424 - DNA, 14 bp, Artificial Sequence
  Designed nucleotides based on the discovery of an optimal set barcoded oligonucleotides for multiplexed RNA sequencing.
tactctcatctagc

SEQ ID NO: 425 - DNA, 14 bp, Artificial Sequence
  Designed nucleotides based on the discovery of an optimal set barcoded oligonucleotides for multiplexed RNA sequencing.
tctctctcaattgg

SEQ ID NO: 426 - DNA, 14 bp, Artificial Sequence
  Designed nucleotides based on the discovery of an optimal set barcoded oligonucleotides for multiplexed RNA sequencing.
ccagtgaggtatac

SEQ ID NO: 427 - DNA, 14 bp, Artificial Sequence
  Designed nucleotides based on the discovery of an optimal set barcoded oligonucleotides for multiplexed RNA sequencing.
ctcctacttaatgc

SEQ ID NO: 428 - DNA, 14 bp, Artificial Sequence
  Designed nucleotides based on the discovery of an optimal set barcoded oligonucleotides for multiplexed RNA sequencing.
ataggccttcgagg

SEQ ID NO: 429 - DNA, 14 bp, Artificial Sequence
  Designed nucleotides based on the discovery of an optimal set barcoded oligonucleotides for multiplexed RNA sequencing.
cattggtggtgtcc

SEQ ID NO: 430 - DNA, 14 bp, Artificial Sequence
  Designed nucleotides based on the discovery of an optimal set barcoded oligonucleotides for multiplexed RNA sequencing.
catactatgtagac

SEQ ID NO: 431 - DNA, 14 bp, Artificial Sequence
  Designed nucleotides based on the discovery of an optimal set barcoded oligonucleotides for multiplexed RNA sequencing.
atggatcgttagga

SEQ ID NO: 432 - DNA, 14 bp, Artificial Sequence
  Designed nucleotides based on the discovery of an optimal set barcoded oligonucleotides for multiplexed RNA sequencing.
ccaaccgaacggca

SEQ ID NO: 433 - DNA, 14 bp, Artificial Sequence
  Designed nucleotides based on the discovery of an optimal set barcoded oligonucleotides for multiplexed RNA sequencing.
acgtacatctatcg

SEQ ID NO: 434 - DNA, 14 bp, Artificial Sequence
  Designed nucleotides based on the discovery of an optimal set barcoded oligonucleotides for multiplexed RNA sequencing.
tacgagtgtctcag

SEQ ID NO: 435 - DNA, 14 bp, Artificial Sequence
  Designed nucleotides based on the discovery of an optimal set barcoded oligonucleotides for multiplexed RNA sequencing.
ataacaacgaagcc

SEQ ID NO: 436 - DNA, 14 bp, Artificial Sequence
  Designed nucleotides based on the discovery of an optimal set barcoded oligonucleotides for multiplexed RNA sequencing.
tacgatagtacagc

SEQ ID NO: 437 - DNA, 14 bp, Artificial Sequence
  Designed nucleotides based on the discovery of an optimal set barcoded oligonucleotides for multiplexed RNA sequencing.
attgcggttaatca

SEQ ID NO: 438 - DNA, 14 bp, Artificial Sequence
  Designed nucleotides based on the discovery of an optimal set barcoded oligonucleotides for multiplexed RNA sequencing.
cagttgcgaagtgg

SEQ ID NO: 439 - DNA, 14 bp, Artificial Sequence
  Designed nucleotides based on the discovery of an optimal set barcoded oligonucleotides for multiplexed RNA sequencing.
ttaacacctcaagg

SEQ ID NO: 440 - DNA, 14 bp, Artificial Sequence
  Designed nucleotides based on the discovery of an optimal set barcoded oligonucleotides for multiplexed RNA sequencing.
cacgacatgcgagc

SEQ ID NO: 441 - DNA, 14 bp, Artificial Sequence
  Designed nucleotides based on the discovery of an optimal set barcoded oligonucleotides for multiplexed RNA sequencing.
tccagaactgaggc

SEQ ID NO: 442 - DNA, 14 bp, Artificial Sequence
  Designed nucleotides based on the discovery of an optimal set barcoded oligonucleotides for multiplexed RNA sequencing.
tattcctagttcga

SEQ ID NO: 443 - DNA, 14 bp, Artificial Sequence
  Designed nucleotides based on the discovery of an optimal set barcoded oligonucleotides for multiplexed RNA sequencing.
aacaccttctccgc

SEQ ID NO: 444 - DNA, 14 bp, Artificial Sequence
  Designed nucleotides based on the discovery of an optimal set barcoded oligonucleotides for multiplexed RNA sequencing.
taacctgttcaaga

SEQ ID NO: 445 - DNA, 14 bp, Artificial Sequence
  Designed nucleotides based on the discovery of an optimal set barcoded oligonucleotides for multiplexed RNA sequencing.
aagctatgtgcaac

SEQ ID NO: 446 - DNA, 14 bp, Artificial Sequence
  Designed nucleotides based on the discovery of an optimal set barcoded oligonucleotides for multiplexed RNA sequencing.
ctcttaatccttga

SEQ ID NO: 447 - DNA, 14 bp, Artificial Sequence
  Designed nucleotides based on the discovery of an optimal set barcoded oligonucleotides for multiplexed RNA sequencing.
atcagcagactagc

SEQ ID NO: 448 - DNA, 14 bp, Artificial Sequence
  Designed nucleotides based on the discovery of an optimal set barcoded oligonucleotides for multiplexed RNA sequencing.
caaggaagcattgg

SEQ ID NO: 449 - DNA, 14 bp, Artificial Sequence
  Designed nucleotides based on the discovery of an optimal set barcoded oligonucleotides for multiplexed RNA sequencing.
acctcgaagttcaa

SEQ ID NO: 450 - DNA, 14 bp, Artificial Sequence
  Designed nucleotides based on the discovery of an optimal set barcoded oligonucleotides for multiplexed RNA sequencing.
atagcactcacacg

SEQ ID NO: 451 - DNA, 14 bp, Artificial Sequence
  Designed nucleotides based on the discovery of an optimal set barcoded oligonucleotides for multiplexed RNA sequencing.
atctagctgcacga

SEQ ID NO: 452 - DNA, 14 bp, Artificial Sequence
  Designed nucleotides based on the discovery of an optimal set barcoded oligonucleotides for multiplexed RNA sequencing.
tctgctgcagagcg

SEQ ID NO: 453 - DNA, 14 bp, Artificial Sequence
  Designed nucleotides based on the discovery of an optimal set barcoded oligonucleotides for multiplexed RNA sequencing.
taacagtccgttca

SEQ ID NO: 454 - DNA, 14 bp, Artificial Sequence
  Designed nucleotides based on the discovery of an optimal set barcoded oligonucleotides for multiplexed RNA sequencing.
catctatctgcgcg

SEQ ID NO: 455 - DNA, 14 bp, Artificial Sequence
  Designed nucleotides based on the discovery of an optimal set barcoded oligonucleotides for multiplexed RNA sequencing.
ttgaccaagattac

SEQ ID NO: 456 - DNA, 14 bp, Artificial Sequence
  Designed nucleotides based on the discovery of an optimal set barcoded oligonucleotides for multiplexed RNA sequencing.
tctcacatcacgag

SEQ ID NO: 457 - DNA, 14 bp, Artificial Sequence
  Designed nucleotides based on the discovery of an optimal set barcoded oligonucleotides for multiplexed RNA sequencing.
acacatctctacga

SEQ ID NO: 458 - DNA, 14 bp, Artificial Sequence
  Designed nucleotides based on the discovery of an optimal set barcoded oligonucleotides for multiplexed RNA sequencing.
aacagaataggcgg

SEQ ID NO: 459 - DNA, 14 bp, Artificial Sequence
  Designed nucleotides based on the discovery of an optimal set barcoded oligonucleotides for multiplexed RNA sequencing.
aacacctaaggaag

SEQ ID NO: 460 - DNA, 14 bp, Artificial Sequence
  Designed nucleotides based on the discovery of an optimal set barcoded oligonucleotides for multiplexed RNA sequencing.
aacacctggttgaa

SEQ ID NO: 461 - DNA, 14 bp, Artificial Sequence
  Designed nucleotides based on the discovery of an optimal set barcoded oligonucleotides for multiplexed RNA sequencing.
aagacgaataggaa

SEQ ID NO: 462 - DNA, 14 bp, Artificial Sequence
  Designed nucleotides based on the discovery of an optimal set barcoded oligonucleotides for multiplexed RNA sequencing.
caagttcagaatgg

SEQ ID NO: 463 - DNA, 14 bp, Artificial Sequence
  Designed nucleotides based on the discovery of an optimal set barcoded oligonucleotides for multiplexed RNA sequencing.
ccaagcttagtgcg

SEQ ID NO: 464 - DNA, 14 bp, Artificial Sequence
  Designed nucleotides based on the discovery of an optimal set barcoded oligonucleotides for multiplexed RNA sequencing.
aacgctcaatgtgg

SEQ ID NO: 465 - DNA, 14 bp, Artificial Sequence
  Designed nucleotides based on the discovery of an optimal set barcoded oligonucleotides for multiplexed RNA sequencing.
tcatctaggtggaa

SEQ ID NO: 466 - DNA, 14 bp, Artificial Sequence
  Designed nucleotides based on the discovery of an optimal set barcoded oligonucleotides for multiplexed RNA sequencing.
cctgtgattacacc

SEQ ID NO: 467 - DNA, 14 bp, Artificial Sequence
  Designed nucleotides based on the discovery of an optimal set barcoded oligonucleotides for multiplexed RNA sequencing.
tcagaacaggttaa

SEQ ID NO: 468 - DNA, 14 bp, Artificial Sequence
  Designed nucleotides based on the discovery of an optimal set barcoded oligonucleotides for multiplexed RNA sequencing.
atattgtaaggtgg

SEQ ID NO: 469 - DNA, 14 bp, Artificial Sequence
  Designed nucleotides based on the discovery of an optimal set barcoded oligonucleotides for multiplexed RNA sequencing.
ttaagtgaccgcgc

SEQ ID NO: 470 - DNA, 14 bp, Artificial Sequence
  Designed nucleotides based on the discovery of an optimal set barcoded oligonucleotides for multiplexed RNA sequencing.
aatcctgaatccaa

SEQ ID NO: 471 - DNA, 14 bp, Artificial Sequence
  Designed nucleotides based on the discovery of an optimal set barcoded oligonucleotides for multiplexed RNA sequencing.
ctatagcgcactac

SEQ ID NO: 472 - DNA, 14 bp, Artificial Sequence
  Designed nucleotides based on the discovery of an optimal set barcoded oligonucleotides for multiplexed RNA sequencing.
tctagacactatcg

SEQ ID NO: 473 - DNA, 14 bp, Artificial Sequence
  Designed nucleotides based on the discovery of an optimal set barcoded oligonucleotides for multiplexed RNA sequencing.
tcaatggcaggccg

SEQ ID NO: 474 - DNA, 14 bp, Artificial Sequence
  Designed nucleotides based on the discovery of an optimal set barcoded oligonucleotides for multiplexed RNA sequencing.
aagtggtattgaca

SEQ ID NO: 475 - DNA, 14 bp, Artificial Sequence
  Designed nucleotides based on the discovery of an optimal set barcoded oligonucleotides for multiplexed RNA sequencing.
ttggacattccggc

SEQ ID NO: 476 - DNA, 14 bp, Artificial Sequence
  Designed nucleotides based on the discovery of an optimal set barcoded oligonucleotides for multiplexed RNA sequencing.
cagcatcgagagcg

SEQ ID NO: 477 - DNA, 14 bp, Artificial Sequence
  Designed nucleotides based on the discovery of an optimal set barcoded oligonucleotides for multiplexed RNA sequencing.
caagaccttggcca

SEQ ID NO: 478 - DNA, 14 bp, Artificial Sequence
  Designed nucleotides based on the discovery of an optimal set barcoded oligonucleotides for multiplexed RNA sequencing.
ccggttcctagtga

SEQ ID NO: 479 - DNA, 14 bp, Artificial Sequence
  Designed nucleotides based on the discovery of an optimal set barcoded oligonucleotides for multiplexed RNA sequencing.
acaacttagcctaa

SEQ ID NO: 480 - DNA, 14 bp, Artificial Sequence
  Designed nucleotides based on the discovery of an optimal set barcoded oligonucleotides for multiplexed RNA sequencing.
tcgtgtcgcatgca

SEQ ID NO: 481 - DNA, 14 bp, Artificial Sequence
  Designed nucleotides based on the discovery of an optimal set barcoded oligonucleotides for multiplexed RNA sequencing.
cacacagacgctcg

SEQ ID NO: 482 - DNA, 14 bp, Artificial Sequence
  Designed nucleotides as a unique molecular identifier enabling the accurate bioinformatic identification of PCR duplicates.
  misc_feature (1)..(9): n is a, c, g, or t
  misc_feature (10)..(14): v is any nucleotide selected from A, C and G
nnnnnnnnnvvvvv

SEQ ID NO: 483 - DNA, 32 bp, Artificial Sequence
  Designed nucleotides as an mRNA capture sequence that specifically hybridizes to mRNA.
  misc_feature (31)..(31): V is any nucleotide selected from A, C and G
  misc_feature (32)..(32): N is any nucleotide selected from A, T, C and G
ttttttttttttttttttttttttttttttvn

SEQ ID NO: 484 - DNA, 22 bp, Artificial Sequence
  Designed nucleotides as a primer.
ctacacgacgctcttccgatct

SEQ ID NO: 485 - DNA, 22 bp, Artificial Sequence
  Designed nucleotides as a primer.
aagcagtggtatcaacgcagag